(19) United States
(12) Patent Application Publication
(10) Pub. No.: US 2023/0255875 A1
(43) Pub. Date: Aug. 17, 2023

(54) COMPOSITIONS COMPRISING TROPOELASTIN CROSSLINKED TO HYALURONIC ACID AND THE METHODS OF USE THEREOF

(71) Applicant: ALLERGAN PHARMACEUTICALS INTERNATIONAL LIMITED, Dublin (IE)

(72) Inventors: Robert DANIELS, Sydney (AU); Christopher K. HEE, Lake Forest, CA (US); Anthony S. WEISS, Sydney (AU); Michal Graff MITZMACHER, Rishon Lezion (IL); Suzanne M. MITHIEUX, Sydney (AU)

(21) Appl. No.: 17/998,763

(22) PCT Filed: May 14, 2021
(86) PCT No.: PCT/IB2021/054177

§ 371 Date: Nov. 14, 2022

Related U.S. Application Data
(60) Provisional application No. 63/025,078, filed on May 14, 2020.

(51) Int. Cl. A61K 8/65; A61K 8/73; A61Q 19/08

(52) U.S. Cl. CPC A61K 8/65; A61K 8/735; A61Q 19/08; A61K 2800/91

(57) ABSTRACT

Soft tissue conditions can be treated by administering a composition comprising tropoelastin including, for example, a composition comprising tropoelastin crosslinked to hyaluronic acid to increase moisture content in the soft tissue. Further, soft tissue conditions can be treated by administering a composition of tropoelastin crosslinked to hyaluronic acid in a ratio of 2:1 or greater that increase moisture content in the soft tissue relative to an otherwise identical composition where the ratio of tropoelastin crosslinked to hyaluronic acid is less than 2:1.

### BACKGROUND

### Field of the Disclosure

The disclosure relates to compositions comprising tropoelastin crosslinked to hyaluronic acid that increase the moisture content in soft tissues and methods of use thereof.

### Description of the Related Art

Skin is an integumentary system organ that interfaces with the environment to protect the body from physical and biological damage. In order to fulfill this role, the skin needs elasticity to respond to and recover from stretch and impact.

Elastin is an essential component of the dermis, providing skin with elasticity and integrity. Elastin and other dermal components are gradually lost through aging, sun damage, and following injury, highlighting a need to replace these components to repair the skin. Aging and tissue injury are associated with degeneration of the extracellular matrix leading to loss of tissue structure and/or function. Loosened skin, relaxed subcutaneous tissue, loss of density of the extracellular matrix, wrinkling, stretch marks and fibrosis are the physical manifestations of the degeneration.

The elastic profile of skin tissues results from a complex process known as elastogenesis involving multiple factors, chief among them tropoelastin (TE), the monomeric subunit of elastin. Elastogenesis is generally understood as referring to a physiological process occurring from late fetal life to early post-natal life whereby elastic fiber is created de novo by cells including fibroblasts, smooth muscle cells and the like from tropoelastin monomers and other relevant factors.

During development, elastin is incorporated into the dermis mainly before birth and in the first few years of life. While there is some elastin deposition in adult tissues, elastogenesis in response to aging or wounding is limited. As such, the tapered production of elastin, coupled with proteolytic loss over time, results in gradual elastin loss during aging. This has a substantial impact on skin tissue, where the combination of decreased elastin and the loss of other macromolecular components like collagen and hyaluronic acid (HA) leads to the reduced structural integrity, moisture content (hydration), and elasticity of skin.

Thus, there exists a strong demand for compositions and methods that restore structural integrity, moisture content (or hydration), and elasticity to skin tissues.

### SUMMARY

The present disclosure provides compositions for treating a soft tissue condition in a subject in need thereof. In some embodiments, the disclosure provides methods of increasing a moisture content in a soft tissue of a subject in need thereof. In further embodiments, the disclosure provides methods and compositions for repairing a soft tissue in a subject in need thereof.

The disclosure provides methods for increasing a moisture content in a soft tissue of a subject in need thereof comprising administering a composition comprising tropoelastin and optionally hyaluronic acid to a soft tissue of the subject.

Additionally, the disclosure provides methods for increasing a moisture content in a soft tissue of a subject in need thereof comprising administering a composition comprising tropoelastin crosslinked to hyaluronic acid to a soft tissue of the subject, wherein the composition increases a moisture content in the soft tissue of the subject as compared to an otherwise identical composition where the tropoelastin is not crosslinked to the hyaluronic acid.

In some embodiments of each or any of the above- or below-mentioned embodiments, the composition comprises tropoelastin crosslinked to the hyaluronic acid without a crosslinker. In embodiments, the tropoelastin is crosslinked to the hyaluronic acid via at least one intermolecular cross-linkage comprising an amide bond between an amine of the tropoelastin and a carboxyl group of the hyaluronic acid.

In some embodiments of each or any of the above- or below-mentioned embodiments, the composition comprises human tropoelastin. In further embodiments, the tropoelastin comprises recombinant tropoelastin, such as recombinant human tropoelastin. In some embodiments, the tropoelastin comprises tropoelastin monomers.

In some embodiments of each or any of the above- or below-mentioned embodiments, the composition comprises tropoelastin in an amount of about 1 mg/ml to about 100 mg/mL. In certain embodiments, tropoelastin is present in an amount of about 10 to about 50 mg/mL. In further embodiments, the tropoelastin is present in an amount of about 30 mg/mL.

In some embodiments of each or any of the above- or below-mentioned embodiments, the composition comprises derivatized hyaluronic acid. In further embodiments, the tropoelastin is crosslinked with the derivatized hyaluronic acid.

In some embodiments of each or any of the above- or below-mentioned embodiments, the composition comprises tropoelastin crosslinked with about 0.1% to about 5% hyaluronic acid. In certain embodiments, the tropoelastin is crosslinked with about 0.5% hyaluronic acid. In further embodiments, hyaluronic acid is present in an amount of about 1 mg/mL to about 15 mg/mL. In still further embodiments, hyaluronic acid is present in an amount of about 10 mg/mL or less, about 5 mg/mL or less, about 4 mg/mL or less, about 3 mg/mL or less, about 2 mg/mL or less, or about 1 mg/mL or less.

In some embodiments of each or any of the above- or below-mentioned embodiments, the composition comprises a ratio of tropoelastin:hyaluronic acid. In some embodiments, the ratio of tropoelastin:hyaluronic acid is about 2:1, about 3:1, about 4:1, about 5:1, about 6:1, about 7:1, about 8:1, about 9:1, or about 10:1.

In some embodiments of each or any of the above- or below-mentioned embodiments, the methods comprise administering a composition comprising tropoelastin crosslinked to hyaluronic acid to a soft tissue of the subject, wherein a ratio of tropoelastin:hyaluronic acid in the composition is about 2:1 or greater, and wherein the second moisture content is increased as compared to a soft tissue administered an otherwise identical composition with a ratio of tropoelastin:hyaluronic acid less than the ratio of tropoelastin:hyaluronic acid in the composition.

In some embodiments of each or any of the above- or below-mentioned embodiments, the ratio of tropoelastin:hyaluronic acid is about 2:1 or greater, and the composition increases a moisture content in the soft tissue of the subject as compared to an otherwise identical composition with a ratio of tropoelastin:hyaluronic acid less than 2:1. In further embodiments, the ratio of tropoelastin:hyaluronic acid is about 2:1 or greater and the amount of hyaluronic acid is less than or equal to about 5 mg/mL.

In some embodiments of each or any of the above- or below-mentioned embodiments, the ratio of tropoelastin:hyaluronic acid is about 6:1 or greater, and the composition increases a moisture content in the soft tissue of the subject as compared to an otherwise identical composition with a ratio of tropoelastin:hyaluronic acid less than 6:1. In further embodiments, the ratio of tropoelastin:hyaluronic acid is about 6:1 or greater and the amount of hyaluronic acid is less than or equal to about 5 mg/mL.

In some embodiments of each or any of the above- or below-mentioned embodiments, the soft tissue is skin. In further embodiments, the skin is very dry skin, dry skin, or hydrated skin before administration of the composition. In still further embodiments, the skin has a capacitance of less than about 90 a.u., less than about 85 a.u., less than about 80 a.u., less than about 75 a.u., less than about 70 a.u., less than about 65 a.u., less than about 60 a.u., less than about 55 a.u., less than about 50 a.u., less than about 45 a.u., less than about 40 a.u., less than about 35 a.u., less than about 30 a.u., less than about 25 a.u., less than about 20 a.u., less than about 15 a.u., less than about 10 a.u., or less than about 5 a.u. before administration of the composition.

In some embodiments of each or any of the above- or below-mentioned embodiments, the disclosure provides methods and compositions for treating a soft tissue condition of the skin. In embodiments, the condition of skin comprises a facial wrinkle, a fine line, thinning skin, aging skin, scar tissue, and a skin depression. In embodiments, methods of the disclosure comprise administering a composition by an injection into the skin. In certain embodiments, a composition is administered to the dermis, hypodermis, or sub-dermis.

In some embodiments of each or any of the above- or below-mentioned embodiments, the methods increase glycosaminoglycan deposition in the soft tissue of a subject in need thereof. In certain embodiments, the methods increase endogenous glycosaminoglycan deposition, such as increased endogenous hyaluronic acid deposition in the soft tissue. In further embodiments, glycosaminoglycan deposition is increased on the surface of cells of a papillary or upper reticular dermis in the skin. In embodiments, glycosaminoglycan deposition is increased by about 2-fold, about 3-fold, about 4-fold, about 5-fold, about 6-fold, about 7-fold, about 8-fold, about 9-fold, or about 10-fold.

In some embodiments of each or any of the above- or below-mentioned embodiments, the methods increase a moisture content in the soft tissue between about 10% to about 80%, or between about 20% to about 50%. In further embodiments, the methods increase a moisture content in the soft tissue by about 20%, about 25%, about 30%, about 35%, about 40%, about 45%, or about 50%.

In some embodiments of each or any of the above- or below-mentioned embodiments, the composition comprising tropoelastin is administered to the soft tissue for a period of time sufficient to increase glycosaminoglycan and/or hyaluronan synthase (e.g., hyaluronan synthase 1 (HAS1), hyaluronan synthase 2 (HAS2), and/or hyaluronan synthase 3 (HAS3)) expression in the soft tissue (e.g., about 2, 3, 4, 5, 6, 7, 8, 9, 10, 10, 30, 40, 50, 60, 70, 80, 90, 100%, or greater increase in expression as compared to an otherwise identical soft tissue not administered the composition).

In some embodiments of each or any of the above- or below-mentioned embodiments, the disclosure provides methods of increasing a moisture content in a soft tissue of a subject in need thereof comprising providing a subject having a soft tissue with a first moisture content and administering a composition comprising tropoelastin crosslinked to hyaluronic acid to the soft tissue in an amount effective to produce a second moisture content in the soft tissue, wherein a ratio of tropoelastin:hyaluronic acid in the composition is about 2:1 or greater, and wherein the second moisture content is increased as compared to a soft tissue administered an otherwise identical composition with a ratio of tropoelastin:hyaluronic acid less than 2:1. In further embodiments, the ratio of tropoelastin:hyaluronic acid in the composition is about 6:1, and the second moisture content is increased as compared to a soft tissue administered an otherwise identical composition with a ratio of tropoelastin:hyaluronic acid less than about 6:1.

In some embodiments of each or any of the above- or below-mentioned embodiments, the methods of the disclosure produce a second moisture content that remains higher than the first moisture content for at least 4 weeks, at least 8 weeks, at least 12 weeks, at least 24 weeks, or at least 48 weeks. In some embodiments of each or any of the above- or below-mentioned embodiments, the methods stimulate collagen synthesis in the soft tissue. In certain embodiments, the methods disclosed herein stimulate elastin synthesis in soft tissue. In other embodiments, the methods of the disclosure stimulate both collagen and elastin synthesis in soft tissue. In some embodiments, the methods of the disclosure increase elasticity of the tissue. In embodiments, the methods of the disclosure lead to cellular infiltration at the site of administration.

The present disclosure also provides methods of stimulating collagen synthesis in the soft tissue in skin of a subject in need thereof, the method comprising administering a composition comprising tropoelastin and optionally hyaluronic acid to a soft tissue of the subject. In an embodiment, the composition has a ratio of tropoelastin:hyaluronic acid of about 2:1 or greater and increases collagen synthesis in the soft tissue of the subject as compared to an otherwise identical composition with a ratio of tropoelastin:hyaluronic acid of less than about 2:1.

The present disclosure also provides methods of stimulating collagen synthesis in the soft tissue in skin of a subject in need thereof, the method comprising administering a composition comprising tropoelastin crosslinked to hyaluronic acid (e.g., a composition with a ratio of tropoelastin:hyaluronic acid of about 2:1 or greater) to a soft tissue of the subject, wherein the composition increases collagen synthesis in the soft tissue of the subject as compared to an otherwise identical composition where the tropoelastin is not crosslinked to the hyaluronic acid.

In some embodiments of each or any of the above- or below-mentioned embodiments, the composition comprises tropoelastin crosslinked to the hyaluronic acid without a crosslinker. In embodiments, the tropoelastin is crosslinked to the hyaluronic acid via at least one intermolecular cross-linkage comprising an amide bond between an amine of the tropoelastin and a carboxyl group of the hyaluronic acid.

In some embodiments of each or any of the above- or below-mentioned embodiments, the composition comprises human tropoelastin. In further embodiments, the tropoelastin comprises recombinant tropoelastin, such as recombinant human tropoelastin. In some embodiments, the tropoelastin comprises tropoelastin monomers.

In some embodiments of each or any of the above- or below-mentioned embodiments, the composition comprises tropoelastin in an amount of about 1 mg/ml to about 100 mg/mL. In certain embodiments, tropoelastin is present in an amount of about 10 to about 50 mg/mL. In further embodiments, the tropoelastin is present in an amount of about 30 mg/mL.

In some embodiments of each or any of the above- or below-mentioned embodiments, the composition comprises derivatized hyaluronic acid.

In some embodiments of each or any of the above- or below-mentioned embodiments, the composition comprises tropoelastin crosslinked with about 0.1% to about 5% hyaluronic acid. In certain embodiments, the tropoelastin is crosslinked with about 0.5% hyaluronic acid. In further embodiments, hyaluronic acid is present in an amount of about 1 mg/mL to about 15 mg/mL. In still further embodiments, hyaluronic acid is present in an amount of about 10 mg/mL or less, about 5 mg/mL or less, about 4 mg/mL or less, about 3 mg/mL or less, about 2 mg/mL or less, or about 1 mg/mL or less.

In some embodiments of each or any of the above- or below-mentioned embodiments, the composition comprises a ratio of tropoelastin:hyaluronic acid. In some embodiments, the ratio of tropoelastin:hyaluronic acid is about 2:1, about 3:1, about 4:1, about 5:1, about 6:1, about 7:1, about 8:1, about 9:1, or about 10:1.

The present disclosure also provides methods of increasing glycosaminoglycan deposition in skin of a subject in need thereof, the method comprising administering a composition comprising tropoelastin and optionally hyaluronic acid to the skin. In an embodiment, the composition has a ratio of tropoelastin:hyaluronic acid of about 2:1 or greater and increases glycosaminoglycan deposition in the soft tissue of the subject as compared to an otherwise identical composition with a ratio of tropoelastin:hyaluronic acid of less than about 2:1.

The present disclosure also provides methods of increasing glycosaminoglycan deposition in skin of a subject in need thereof, the method comprising administering a composition comprising tropoelastin crosslinked to hyaluronic acid (e.g., a composition with a ratio of tropoelastin:hyaluronic acid of about 2:1 or greater) to the skin, wherein glycosaminoglycan deposition is increased as compared to a soft tissue administered an otherwise identical composition where the tropoelastin is not crosslinked to the hyaluronic acid.

In some embodiments of each or any of the above- or below-mentioned embodiments, the composition comprises tropoelastin crosslinked to the hyaluronic acid without a crosslinker. In embodiments, the tropoelastin is crosslinked to the hyaluronic acid via at least one intermolecular cross-linkage comprising an amide bond between an amine of the tropoelastin and a carboxyl group of the hyaluronic acid.

In some embodiments of each or any of the above- or below-mentioned embodiments, the composition comprises human tropoelastin. In further embodiments, the tropoelastin comprises recombinant tropoelastin, such as recombinant human tropoelastin. In some embodiments, the tropoelastin comprises tropoelastin monomers.

In some embodiments of each or any of the above- or below-mentioned embodiments, the composition comprises tropoelastin in an amount of about 1 mg/ml to about 100 mg/mL. In certain embodiments, tropoelastin is present in an amount of about 10 to about 50 mg/mL. In further embodiments, the tropoelastin is present in an amount of about 30 mg/mL.

In some embodiments of each or any of the above- or below-mentioned embodiments, the composition comprises derivatized hyaluronic acid.

In some embodiments of each or any of the above- or below-mentioned embodiments, the composition comprises tropoelastin crosslinked with about 0.1% to about 5% hyaluronic acid. In certain embodiments, the tropoelastin is crosslinked with about 0.5% hyaluronic acid. In further embodiments, hyaluronic acid is present in an amount of about 1 mg/mL to about 15 mg/mL. In still further embodiments, hyaluronic acid is present in an amount of about 10 mg/mL or less, about 5 mg/mL or less, about 4 mg/mL or less, about 3 mg/mL or less, about 2 mg/mL or less, or about 1 mg/mL or less.

In some embodiments of each or any of the above- or below-mentioned embodiments, the composition comprises a ratio of tropoelastin:hyaluronic acid. In some embodiments, the ratio of tropoelastin:hyaluronic acid is about 2:1, about 3:1, about 4:1, about 5:1, about 6:1, about 7:1, about 8:1, about 9:1, or about 10:1.

The present disclosure also provides methods of increasing hyaluronan synthase expression (e.g., hyaluronan synthase 1 (HAS1), hyaluronan synthase 2 (HAS2), and/or hyaluronan synthase 3 (HAS3)) in the skin of a subject of a subject in need thereof, the method comprising administering a composition comprising tropoelastin and optionally hyaluronic acid to the skin. In an embodiment, the composition has a ratio of tropoelastin:hyaluronic acid of about 2:1 or greater and increases hyaluronan synthase expression in the soft tissue of the subject as compared to an otherwise identical composition with a ratio of tropoelastin:hyaluronic acid of less than about 2:1. In some embodiments, hyaluronan synthase expression is increased by about 2-fold, about 3-fold, about 4-fold, about 5-fold, about 6-fold, about 7-fold, about 8-fold, about 9-fold, or about 10-fold relative to hyaluronan synthase expression in untreated skin.

The present disclosure also provides methods of increasing hyaluronan synthase expression (e.g., hyaluronan synthase 1 (HAS1), hyaluronan synthase 2 (HAS2), and/or hyaluronan synthase 3 (HAS3)) in the skin of a subject of a subject in need thereof, the method comprising administering a composition comprising tropoelastin crosslinked to hyaluronic acid (e.g., a composition with a ratio of tropoelastin:hyaluronic acid of about 2:1 or greater) to the skin, and wherein hyaluronan synthase expression is increased as compared to a soft tissue administered an otherwise identical composition where the tropoelastin is in monomeric form (e.g., a composition with a ratio of tropoelastin:hyaluronic acid of less than about 2:1). In some embodiments, hyaluronan synthase expression is increased by about 2-fold, about 3-fold, about 4-fold, about 5-fold, about 6-fold, about 7-fold, about 8-fold, about 9-fold, or about 10-fold relative to hyaluronan synthase expression in untreated skin.

In some embodiments of each or any of the above- or below-mentioned embodiments, the composition comprises tropoelastin crosslinked to the hyaluronic acid without a crosslinker. In embodiments, the tropoelastin is crosslinked to the hyaluronic acid via at least one intermolecular cross-linkage comprising an amide bond between an amine of the tropoelastin and a carboxyl group of the hyaluronic acid.

In some embodiments of each or any of the above- or below-mentioned embodiments, the composition comprises human tropoelastin. In further embodiments, the tropoelastin comprises recombinant tropoelastin, such as recombinant human tropoelastin. In some embodiments, the tropoelastin comprises tropoelastin monomers.

In some embodiments of each or any of the above- or below-mentioned embodiments, the composition comprises tropoelastin in an amount of about 1 mg/ml to about 100 mg/mL. In certain embodiments, tropoelastin is present in an amount of about 10 to about 50 mg/mL. In further embodiments, the tropoelastin is present in an amount of about 30 mg/mL.

In some embodiments of each or any of the above- or below-mentioned embodiments, the composition comprises derivatized hyaluronic acid.

In some embodiments of each or any of the above- or below-mentioned embodiments, the composition comprises tropoelastin crosslinked with about 0.1% to about 5% hyaluronic acid. In certain embodiments, the tropoelastin is crosslinked with about 0.5% hyaluronic acid. In further embodiments, hyaluronic acid is present in an amount of about 1 mg/mL to about 15 mg/mL. In still further embodiments, hyaluronic acid is present in an amount of about 10 mg/mL or less, about 5 mg/mL or less, about 4 mg/mL or less, about 3 mg/mL or less, about 2 mg/mL or less, or about 1 mg/mL or less.

In some embodiments of each or any of the above- or below-mentioned embodiments, the composition comprises a ratio of tropoelastin:hyaluronic acid. In some embodiments, the ratio of tropoelastin:hyaluronic acid is about 2:1, about 3:1, about 4:1, about 5:1, about 6:1, about 7:1, about 8:1, about 9:1, or about 10:1.

The present disclosure also provides methods of improving skin composition (e.g., skin brightness, skin elasticity, and/or an increase in extracellular matrix proteins) in a subject in need thereof, the method comprising administering a composition comprising tropoelastin and optionally hyaluronic acid to the skin. In an embodiment, the composition has a ratio of tropoelastin:hyaluronic acid of about 2:1 or greater and improves skin composition of the subject as compared to an otherwise identical composition with a ratio of tropoelastin:hyaluronic acid of less than about 2:1.

The present disclosure also provides methods of improving skin composition (e.g., skin brightness, skin elasticity, and/or an increase in extracellular matrix proteins) in a subject in need thereof, the method comprising administering a composition comprising tropoelastin crosslinked to hyaluronic acid (e.g., a composition with a ratio of tropoelastin:hyaluronic acid of about 2:1 or greater) to the skin, wherein skin composition is improved as compared to a soft tissue administered an otherwise identical composition where the tropoelastin is not crosslinked to the hyaluronic acid.

In some embodiments of each or any of the above- or below-mentioned embodiments, the composition comprises tropoelastin crosslinked to the hyaluronic acid without a crosslinker. In embodiments, the tropoelastin is crosslinked to the hyaluronic acid via at least one intermolecular cross-linkage comprising an amide bond between an amine of the tropoelastin and a carboxyl group of the hyaluronic acid.

In some embodiments of each or any of the above- or below-mentioned embodiments, the composition comprises human tropoelastin. In further embodiments, the tropoelastin comprises recombinant tropoelastin, such as recombinant human tropoelastin. In some embodiments, the tropoelastin comprises tropoelastin monomers.

In some embodiments of each or any of the above- or below-mentioned embodiments, the composition comprises tropoelastin in an amount of about 1 mg/ml to about 100 mg/mL. In certain embodiments, tropoelastin is present in an amount of about 10 to about 50 mg/mL. In further embodiments, the tropoelastin is present in an amount of about 30 mg/mL.

In some embodiments of each or any of the above- or below-mentioned embodiments, the composition comprises derivatized hyaluronic acid.

In some embodiments of each or any of the above- or below-mentioned embodiments, the composition comprises tropoelastin crosslinked with about 0.1% to about 5% hyaluronic acid. In certain embodiments, the tropoelastin is crosslinked with about 0.5% hyaluronic acid. In further embodiments, hyaluronic acid is present in an amount of about 1 mg/mL to about 15 mg/mL. In still further embodiments, hyaluronic acid is present in an amount of about 10 mg/mL or less, about 5 mg/mL or less, about 4 mg/mL or less, about 3 mg/mL or less, about 2 mg/mL or less, or about 1 mg/mL or less.

In some embodiments of each or any of the above- or below-mentioned embodiments, the composition comprises a ratio of tropoelastin:hyaluronic acid. In some embodiments, the ratio of tropoelastin:hyaluronic acid is about 2:1, about 3:1, about 4:1, about 5:1, about 6:1, about 7:1, about 8:1, about 9:1, or about 10:1.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1A shows moisture content of skin explants at 1, 2, 5, and 7 days after intradermal injection of control; 12 mg/mL hyaluronic acid (HA) only; 10 mg/mL recombinant human tropoelastin (rhTE) monomers mixed (i.e. uncrosslinked) with 12 mg/mL HA (F7); 30 mg/mL rhTE monomers mixed (i.e. uncrosslinked) with 12 mg/mL HA (F6); 10 mg/mL rhTE crosslinked with 0.5% dHA in phosphate-buffered saline (PBS) (F9), and 30 mg/mL rhTE crosslinked with 0.5% dHA in PBS (F8). FIG. 1B shows immunofluorescence staining of acidic glycosaminoglycans (GAGs) in the papillary dermis at 5 and 7 days following injections. FIG. 1C shows immunofluorescence staining of acidic GAGs in the upper reticular dermis at 5 and 7 days following injections.

FIGS. 2A-2F show representative hematoxylin and eosin (H&E) images of cross-sections from rat skin biopsies at 2 weeks following intradermal injection. FIG. 2A shows results following intradermal injection of 10 mg/mL rhTE crosslinked with 0.5% derivatized hyaluronic acid (dHA) in PBS. FIG. 2B shows results following intradermal injection 30 mg/mL rhTE crosslinked with 0.5% dHA in PBS. FIG. 2C shows results following intradermal injection of 10 mg/mL rhTE monomers mixed (i.e. uncrosslinked) with 12 mg/mL HYC-24L+. FIG. 2D shows results following intradermal injection 30 mg/mL rhTE monomers mixed (i.e. uncrosslinked) with 12 mg/mL HYC-24L+. FIG. 2E shows results following intradermal injection of 12 mg/mL HYC-24L+. FIG. 2F shows results following intradermal injection of saline control. Collagenous tissue appears as pink fibers, HA appears as fields of light to dark purple, and cell nuclei as punctate dark purple staining.

FIGS. 3A-3F show representative images (100×) of immunofluorescent double labeling of recombinant human elastin (red) and rat elastin (green) of cross-sections from skin biopsies at 8 weeks following intradermal injection. FIG. 3A shows results following intradermal injection of 10 mg/mL rhTE crosslinked with 0.5% dHA in PBS. FIG. 3B shows results following intradermal injection 30 mg/mL rhTE crosslinked with 0.5% dHA in PBS. FIG. 3C shows results following intradermal injection of 10 mg/mL rhTE monomers mixed (i.e. uncrosslinked) with 12 mg/mL HYC-24L+. FIG. 3D shows results following intradermal injection 30 mg/mL rhTE monomers mixed (i.e. uncrosslinked) with 12 mg/mL HYC-24L+. FIG. 3E shows results following intradermal injection of 12 mg/mL HYC-24L+. FIG. 3F shows results following intradermal injection of saline control. Nuclei are counterstained with DAPI (blue).

FIG. 4 shows representative confocal microscopy images of fibroblasts incubated in the absence or presence of L-ascorbic acid 2-phosphate sesquimagnesium salt (APM), with and without low-dose (x1) and high-dose (x2) tropoelastin. Z-stack images of fibroblast cultures from top layers (layer 1) to bottom layers (layer 4) allowed for detection of elastin (red), collagen type I (green), and nuclei (blue) in different layers.

FIGS. 5A-5B show qPCR analysis of hyaluronan synthase 1 (HAS1) mRNA. FIG. 5A shows HAS1 levels at 24 hours and FIG. 5B shows HAS1 levels at 120 hours following injection of skin patches with test formulations (F1—F5, see Table 1). All values are mean (SD). *P<0.05 compared with control.

FIGS. 6A-6D show glycosaminoglycan (GAG) deposition by dermal fibroblasts. FIG. 6A shows Alcian blue stained neonatal and FIG. 6B shows Alcian blue stained adult dermal fibroblast cultures 7 days after the addition of hyaluronic acid (HA), tropoelastin (TE), or both. FIG. 6C (neonatal) and FIG. 6D (adult) show quantitation of experiments exemplified in FIGS. 6A and 6B. Controls without added HA or TE are labeled “cells.”

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

The present disclosure provides methods and compositions for treating or repairing a soft tissue condition in a subject in need thereof. As used herein, “soft tissue” means tissue that connects, supports, or surrounds bone and internal organs. For example, soft tissues include skin, muscle, fat, tendon, and fascia. A soft tissue “condition” comprises a disease, disorder, pathology, non-pathological condition, or departure from a state of homeostasis.

In embodiments, the disclosure provides methods of increasing a moisture content in a soft tissue of a subject in need thereof. As used herein, “moisture content” means a hydration level in a soft tissue comprising the amount of water present in the cells and/or extracellular space of the tissue. Methods of measuring the moisture content of soft tissue are known in the art. For example, in the context of the present disclosure, the moisture content, or hydration level, of skin may be measured based on changes in dielectric constant due to skin surface hydration. Preferably, skin hydration is measured with a CORNEOMETER®, a hand-held probe from Courage+Khazaka Electronics GmbH (Cologne, Germany) (see Examples).

As used herein, “increasing a moisture content” or “increased moisture content” means increasing the water content or hydration level present in the cells and/or extracellular space of the tissue including, for example, relative to a comparator tissue. In some embodiments, a comparator tissue may comprise a soft tissue before treatment with a composition of the disclosure. In some embodiments a comparator may comprise a soft tissue that is untreated, or is administered a composition other than those disclosed herein. In some embodiments, methods of the disclosure increase a moisture content in the soft tissue between about 10% to about 80%, or between about 20% to about 50%. In further embodiments, methods of the disclosure increase a moisture content in the soft tissue by about 1%, about 2%, about 3%, about 4%, about 5%, about 6%, about 7%, about 8%, about 9%, about 10%, about 20%, about 30%, about 40%, about 50%, about 60%, about 70%, about 80%, or about 90%. In certain embodiments, a composition of the disclosure increases a moisture content by about 30% relative to a soft tissue that is untreated, or is administered a composition other than those disclosed herein.

The present disclosure provides methods for increasing a moisture content in a soft tissue comprising administering a composition comprising tropoelastin and optionally hyaluronic acid to a soft tissue of the subject. In an embodiment, the tropoelastin is crosslinked to the hyaluronic acid. In a further embodiment, the tropoelastin is crosslinked with derivatized hyaluronic acid. In another embodiment, the tropoelastin is human tropoelastin. In further embodiments, the tropoelastin comprises recombinant tropoelastin, such as recombinant human tropoelastin. In other embodiments, the tropoelastin comprises tropoelastin monomers.

In some embodiments, the methods of the disclosure comprise a composition comprising tropoelastin present in an amount of about 1 mg/ml to about 100 mg/mL. For example, in embodiments, tropoelastin is present in in an amount of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, or about 100 mg/ml. In certain embodiments, tropoelastin is present in an amount of about 30 mg/mL.

The amount and concentration of tropoelastin to be administered is dependent on both the area and volume of tissue to be treated, the content in the tissue normally; and, the level of increased moisture content required. In embodiments, tropoelastin will be administered to the tissue in an amount of about 1 μg to about 1 mg per each cm3 of tissue. For skin this may be calculated as about 1 μg to about 1 mg of cm2. Other amounts which may be administered include about 0.1 μg to 1 about 0 mg per each cm³ of tissue, about 1 mg to about 20 mg per each cm3 of tissue, or about 1 mg to about 100 mg per cm3 of tissue. In certain embodiments the amounts administered may be less than about 0.1 μg or more than about 100 mg per cm³ of tissue. The concentration of tropoelastin in the administered composition may vary to enable the required amounts of tropoelastin to be administered.

In embodiments, the tropoelastin of the disclosure is substantially equivalent to an isoform of tropoelastin which occurs naturally in the tissue to be treated. In addition, the tropoelastin should be provided in a form which is substantially devoid of impurities. Fragments of tropoelastin, i.e. truncated forms of a tropoelastin isoform that arise unintentionally through tropoelastin manufacture may be regarded as an impurity in this context. In certain embodiments, tropoelastin incorporated into the treatment formulation will be at least 65% of the length of the relevant full length tropoelastin isoform, more preferably 80% of the relevant full length tropoelastin isoform. In other embodiments the tropoelastin will be more than 85%, more than 90%, or more than 95% full length.

In embodiments, the tropoelastin of the disclosure is modified to reduce protease degradation. For example, protein species may be selected as described in WO 2000/04043 to the extent that they remain substantially full length tropoelastin species naturally found in the tissue to be treated. Alternatively, the treatment formulations may incorporate protease inhibitors or molecules which block signalling pathways known to increase protease expression. Such molecules include serine protease inhibitors, matrix metalloproteinase inhibitors, galactosides such as lactose, inhibitory antibodies and small molecule inhibitors of elastin signalling.

In embodiments comprising treating a human subject, the tropoelastin has the sequence of a tropoelastin isoform that is expressed in humans. In some embodiments, the isoform may be selected from the group consisting of SHEL (see, WO 1994/14958) and SHELδ26A (see, WO 1999/03886) and protease resistant derivatives of these isoforms (see WO 2000/0403). In certain embodiments, the tropoelastin isoform is SHELδ26A.

In certain embodiments the tropoelastin has a specified degree of purity with respect to the amount of tropoelastin in a composition for administration, as compared with amounts of other proteins or molecules in the composition. In one embodiment, the tropoelastin is in a composition that has at least about 75% purity, preferably about 85% purity, more preferably more than about 90, or about 95% purity. It will be understood that in certain embodiments the tropoelastin may be provided in the form of a composition that consists of, or consists essentially of tropoelastin, preferably a full-length isoform of tropoelastin. Finally, cells are unable to utilize tropoelastin to form elastic fiber if the tropoelastin has already been substantially intra-molecularly cross linked.

Typically, the composition for administration including tropoelastin does not contain exogenous factors for elastic fiber formation, especially lysyl oxidase.

In certain embodiments the tropoelastin is provided according to a treatment regime in a substantially monomeric form.

In certain embodiments the tropoelastin is provided according to a treatment regime in a form substantially lacking intra-molecular cross-links.

In certain embodiments the tropoelastin is provided according to a treatment regime in a composition that consists of tropoelastin and a solvent for the tropoelastin, such as an aqueous solution.

In embodiments, a composition of the disclosure comprises one or more compounds that increase the utilization of tropoelastin. Exemplary compounds that increase the utilization of tropoelastin include diclofenac, Lys'lastine, amino acids (e.g., Gly, Val, Ala,), vitamins (e.g., C, E), sunscreen, and chemical enhancers.

In some embodiments, the methods of the disclosure comprise tropoelastin crosslinked to hyaluronic acid. In embodiments, the disclosure provides methods comprising administering a composition comprising tropoelastin crosslinked to hyaluronic acid to a soft tissue of the subject, wherein the composition increases a moisture content in the soft tissue of the subject as compared to an otherwise identical composition where the tropoelastin is not crosslinked to the hyaluronic acid.

In some embodiments, the methods of the disclosure comprise a composition comprising tropoelastin crosslinked to the hyaluronic acid without a crosslinker. For example, in embodiments the tropoelastin is crosslinked to the hyaluronic acid without a crosslinking agent, such as an enzymatic crosslinking agent. In certain embodiments, a composition of the disclosure comprises tropoelastin crosslinked to hyaluronic acid without treatment under alkaline conditions. In some embodiments, the tropoelastin is crosslinked to the hyaluronic acid via at least one intermolecular cross-linkage comprising an amide bond between an amine of the tropoelastin and a carboxyl group of the hyaluronic acid.

In certain embodiments, a composition comprising tropoelastin is crosslinked with about 0.1% (w/v) to about 5% (w/v) hyaluronic acid. In some embodiments, the hyaluronic acid comprises about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), about 3.0% (w/v), about 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4.0% (w/v), about 4.1% (w/v), about 4.2% (w/v), about 4.3% (w/v), about 4.4% (w/v), about 4.5% (w/v), about 4.6% (w/v), about 4.7% (w/v), about 4.8% (w/v), about 4.9% (w/v), or about 5.0% (w/v). In certain embodiments, the tropoelastin is crosslinked with about 0.5% (w/v) hyaluronic acid.

In further embodiments, the tropoelastin is crosslinked with hyaluronic acid present in an amount of about 1 mg/mL to about 15 mg/mL. In still further embodiments, the tropoelastin is crosslinked with hyaluronic acid present in an amount of about 10 mg/mL or less, about 5 mg/mL or less, about 4 mg/mL or less, about 3 mg/mL or less, about 2 mg/mL or less, or about 1 mg/mL or less. In certain embodiments, the tropoelastin is crosslinked with hyaluronic acid present in an amount of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, or about 15 mg/ml.

In certain embodiments, the tropoelastin in the composition may be crosslinked to derivatized hyaluronic acid.

In some embodiments, the methods of the disclosure comprise a composition comprising a ratio of tropoelastin to hyaluronic acid (“tropoelastin:hyaluronic acid”). As used herein, a “ratio of tropoelastin:hyaluronic acid” comprises the ratio of the weight/volume concentration of each component in a composition. For example, a ratio of about 6:1 tropoelastin:hyaluronic acid comprises a composition with about 30 mg/mL (or about 3% (w/v)) tropoelastin and about 5 mg/mL (or about 0.5% (w/v)) hyaluronic acid. In some embodiments, the ratio of tropoelastin:hyaluronic acid is about 2:1, about 3:1, about 4:1, about 5:1, about 6:1, about 7:1, about 8:1, about 9:1, or about 10:1.

In some embodiments, the ratio of tropoelastin:hyaluronic acid is about 2:1 or greater, and the composition increases a moisture content in the soft tissue of the subject as compared to an otherwise identical composition with a ratio of tropoelastin:hyaluronic acid less than about 2:1. Thus, embodiments of the methods disclosed herein employ compositions that result in greater increases in moisture content in soft tissues despite having lower amounts of hyaluronic acid relative to a comparator composition having a higher amount of hyaluronic acid. In some embodiments, the ratio of tropoelastin:hyaluronic acid is about 2:1 or greater and the amount of hyaluronic acid is less than or equal to about 5 mg/mL, wherein the composition results in greater increases in moisture content in soft tissues relative to a composition with more than 5 mg/mL hyaluronic acid. In embodiments, the ratio of tropoelastin:hyaluronic acid is about 2:1, the amount of tropoelastin is about 10 mg/mL, and the amount of hyaluronic acid is about 5 mg/mL.

In certain embodiments, the ratio of tropoelastin:hyaluronic acid is about 3:1 or greater, and the composition increases a moisture content in the soft tissue of the subject as compared to an otherwise identical composition with a ratio of tropoelastin:hyaluronic acid less than 3:1. In some embodiments, the ratio of tropoelastin:hyaluronic acid is about 3:1 or greater and the amount of hyaluronic acid is less than or equal to about 5 mg/mL, wherein the composition results in greater increases in moisture content in soft tissues relative to a composition with more than 5 mg/mL hyaluronic acid. In embodiments, the ratio of tropoelastin:hyaluronic acid is about 3:1, the amount of tropoelastin is about 15 mg/mL, and the amount of hyaluronic acid is about 5 mg/mL.

In certain embodiments, the ratio of tropoelastin:hyaluronic acid is about 4:1 or greater, and the composition increases a moisture content in the soft tissue of the subject as compared to an otherwise identical composition with a ratio of tropoelastin:hyaluronic acid less than 4:1. In some embodiments, the ratio of tropoelastin:hyaluronic acid is about 4:1 or greater and the amount of hyaluronic acid is less than or equal to about 5 mg/mL, wherein the composition results in greater increases in moisture content in soft tissues relative to a composition with more than 5 mg/mL hyaluronic acid. In embodiments, the ratio of tropoelastin:hyaluronic acid is about 4:1, the amount of tropoelastin is about 20 mg/mL, and the amount of hyaluronic acid is about 5 mg/mL.

In certain embodiments, the ratio of tropoelastin:hyaluronic acid is about 5:1 or greater, and the composition increases a moisture content in the soft tissue of the subject as compared to an otherwise identical composition with a ratio of tropoelastin:hyaluronic acid less than 5:1. In some embodiments, the ratio of tropoelastin:hyaluronic acid is about 5:1 or greater and the amount of hyaluronic acid is less than or equal to about 5 mg/mL, wherein the composition results in greater increases in moisture content in soft tissues relative to a composition with more than 5 mg/mL hyaluronic acid. In embodiments, the ratio of tropoelastin:hyaluronic acid is about 5:1, the amount of tropoelastin is about 25 mg/mL, and the amount of hyaluronic acid is about 5 mg/mL.

In certain embodiments, the ratio of tropoelastin:hyaluronic acid is about 6:1 or greater, and the composition increases a moisture content in the soft tissue of the subject as compared to an otherwise identical composition with a ratio of tropoelastin:hyaluronic acid less than 6:1. In some embodiments, the ratio of tropoelastin:hyaluronic acid is about 6:1 or greater and the amount of hyaluronic acid is less than or equal to about 5 mg/mL, wherein the composition results in greater increases in moisture content in soft tissues relative to a composition with more than 5 mg/mL hyaluronic acid. In embodiments, the ratio of tropoelastin:hyaluronic acid is about 6:1, the amount of tropoelastin is about 30 mg/mL, and the amount of hyaluronic acid is about 5 mg/mL.

In certain embodiments, the ratio of tropoelastin:hyaluronic acid is about 7:1 or greater, and the composition increases a moisture content in the soft tissue of the subject as compared to an otherwise identical composition with a ratio of tropoelastin:hyaluronic acid less than 7:1. In some embodiments, the ratio of tropoelastin:hyaluronic acid is about 7:1 or greater and the amount of hyaluronic acid is less than or equal to about 5 mg/mL, wherein the composition results in greater increases in moisture content in soft tissues relative to a composition with more than 5 mg/mL hyaluronic acid. In embodiments, the ratio of tropoelastin:hyaluronic acid is about 7:1, the amount of tropoelastin is about 35 mg/mL, and the amount of hyaluronic acid is about 5 mg/mL.

In certain embodiments, the ratio of tropoelastin:hyaluronic acid is about 8:1 or greater, and the composition increases a moisture content in the soft tissue of the subject as compared to an otherwise identical composition with a ratio of tropoelastin:hyaluronic acid less than 8:1. In some embodiments, the ratio of tropoelastin:hyaluronic acid is about 8:1 or greater and the amount of hyaluronic acid is less than or equal to about 5 mg/mL, wherein the composition results in greater increases in moisture content in soft tissues relative to a composition with more than 5 mg/mL hyaluronic acid. In embodiments, the ratio of tropoelastin:hyaluronic acid is about 8:1, the amount of tropoelastin is about 40 mg/mL, and the amount of hyaluronic acid is about 5 mg/mL.

In some embodiments, the soft tissue is skin. In further embodiments, the skin is very dry skin, dry skin, or hydrated skin before administration of the composition. In still further embodiments, the skin has a capacitance of less than about 90 a.u., less than about 85 a.u., less than about 80 a.u., less than about 75 a.u., less than about 70 a.u., less than about 65 a.u., less than about 60 a.u., less than about 55 a.u., less than about 50 a.u., less than about 45 a.u., less than about 40 a.u., less than about 35 a.u., less than about 30 a.u., less than about 25 a.u., less than about 20 a.u., less than about 15 a.u., less than about 10 a.u., or less than about 5 a.u. before administration of the composition.

In other embodiments, the skin has a capacitance of 30-60 a.u. (e.g., very dry skin), 60-70 a.u. (e.g., dry skin), or 70-90 a.u. (e.g., hydrated skin) before administration of the composition including, as measured with a Corneometer (see, Zuang et al. (1997) J. Appl. Cosmetol. 15, 95-102). In further embodiments, the skin has increased capacitance after administration of the composition including a 5%, 10%, 15%, 20%, 25%, 30%, 35%, 40%, 45%, 50%, 55%, 60%, 65%, 70%, 75%, 80%, 85%, 90%, 95%, 100%, or greater increase in capacitance. In some embodiments, the skin is identified as very dry skin before administration of the composition and identified as dry skin, hydrated skin, or very moist skin after administration of the composition. In other embodiments, the skin is identified as dry skin before administration of the composition and identified as hydrated skin or very moist skin after administration of the composition. In some embodiments, the skin is identified as hydrated skin before administration of the composition and identified as very moist skin after administration of the composition.

In some embodiments, the disclosure provides methods and compositions for treating a soft tissue condition of the skin. In embodiments, the condition of skin comprises a facial wrinkle, a fine line, thinning skin, aging skin, scar tissue, and a skin depression. In embodiments, methods of the disclosure comprise administering a composition by an injection into the skin. In certain embodiments, a composition is administered to the dermis, hypodermis, or sub-dermis.

In some embodiments, the tissue is skin tissue, such as skin tissue in an individual of at least about 20 years, about 20 to about 50 years of age, or about 30 to about 60 years of age or older.

In some embodiments, the skin tissue treated according to the disclosure may be characterized by a breakage or fragmentation of elastic fibers at the junction of the dermis and epidermis, and a low moisture content relative to healthy skin tissue. The skin tissue may be photo-aged tissue. The skin tissue may present with one or more of the following features: loosened skin, relaxed subcutaneous tissue, loss of density of the extracellular matrix, wrinkling and stretch marks. The skin tissue is preferably located on the face, neck or upper or lower limb.

In some embodiments, the compositions of the disclosure are administered by injection. Where the tissue is skin, it is preferred that the composition is administered to the dermis. In certain embodiments, the composition is administered by injection into the mid- to deep-dermis by fine needle injection. The injection may be made using a hypodermic needle with a gauge of 25 G, preferably, 27G or less, more preferably 30 G or 31 G. The injection may be made using a single syringe and needle by manual application of the treatment to the skin. In certain embodiments, a single treatment may include multiple injections into a treatment area. Where each treatment requires multiple injections, these may be spaced from 1 mm to 3 cm apart.

In certain embodiments, the injection may be made using a device which enables automated injection into the skin dermis such as a Mesotherapy gun, or an assisted injection device such as the artiste injection device or the anteis injection device. In certain embodiments, the syringe or automated injection device may be used with an adaptor to enable multiple needles to be attached so that more than one injection can be applied at a time. In certain embodiments, the treatment may be applied using a solid needle system such as a dermal roller, or dermapen needling system (e.g. as described by Kalluri, H. et al 2011, AAPS Journal 13:473-4841).

There may be a period of about 1 day to 6 months between each treatment. Typical periods between each treatment may include about 1 to about 7 days, about 7 to about 14 days, about 21 to about 28 days, about 28 to about 49 days, and about 49 to about 100 days. There may be about 1 to about 24, or about 3 to about 6 treatments in total. Generally, the period of treatment is no more than about 1 year, preferably from about 3 weeks to about 6 months, preferably about 1 to about 3 months. In embodiments, there is a period between each treatment of about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 25 days, about 30 days, about 35 days, about 40 days, about 45 days, about 50 days, about 55 days, about 60 days, about 65 days, about 70 days, about 75 days, about 80 days, about 85 days, about 90 days, about 95 days, or about 100 days.

In some embodiments, sites of treatment include those near, about, within or adjacent to cheeks, the eyes, neck, décolletage, hands, scarred tissue, stretch marks.

In some embodiments, additional components are included in the composition to assist in the treatment or repair of a soft tissue condition, and/or increase in moisture content in the soft tissue. For example, for the treatment of skin, additional components may be incorporated into the formulation that assists in the recruitment or proliferation of fibroblast cells at the treatment site. Such components include the epidermal growth factor family, transforming growth factor beta family, fibroblast growth factor family, vascular endothelial growth factor, granulocyte macrophage colony stimulating factor, platelet-derived growth factor, connective tissue growth factor, interleukin family, and tumor necrosis factor-a family.

In certain embodiments, the treatment regime may additionally include the topical application of substances capable of augmenting, treating, or repairing a soft tissue condition or increasing the moisture content of the soft tissue. Such substances would be well known to those skilled in the art and may include but are not limited to a dill extract to stimulate lysyl oxidase expression (Cenizo et al 2006 Exp. Dermatol. 15:574-81); and, copper and/or zinc-based creams to reduce elastic fiber breakdown (Mahoney et al 2009 Exp. Dermatol. 18:205-211).

In certain embodiments the treatment may also include the delivery of cells to the treatment site with the tropoelastin. By way of example for the treatment of skin, fibroblasts may be included in the treatment formulation or procedure to aid the synthesis of elastic fiber at the treatment site. The fibroblast cells may be sourced from an allogeneic source such as neonatal foreskin or sourced by biopsy of a non-visible skin site (e.g. behind the ear) and used as an autologous treatment.

In some embodiments, the disclosure provides a method of treating a condition of the skin of a subject, the method comprising providing an subject with a skin condition, defining a treatment area on the skin of the subject, wherein the treatment area is an area of skin in which moisture content is to be increased, injecting a composition comprising crosslinked tropoelastin and hyaluronic acid within the treatment area so as to establish an amount crosslinked tropoelastin and hyaluronic acid within the treatment area that is increased relative to skin outside the treatment area, and maintaining the amount of crosslinked tropoelastin and hyaluronic acid in the treatment area for a pre-determined period of time, thereby increasing the moisture content in the skin of the subject.

In embodiments, the crosslinked tropoelastin and hyaluronic acid is maintained in the treatment area for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 25 days, about 30 days, about 35 days, about 40 days, about 45 days, about 50 days, about 55 days, about 60 days, about 65 days, about 70 days, about 75 days, about 80 days, about 85 days, about 90 days, about 95 days, or about 100 days.

In certain embodiments, to ensure the tropoelastin is delivered in a form which can be utilized by cells as a substrate for the construction of elastic fiber and remain at the treatment site for a sufficient period of time for this to occur, the treatment is applied to the site on repeated occasions. In certain embodiments each tissue site to be treated will receive the three treatments of the product, from about 1 to about 24, or about 2 to about 12 or about 3 to about 6 weeks apart. The treatment may consist of multiple injections across the area to be treated, each approximately 10 mm apart in a grid formation. The treatment may be administered using a fine gauge needle, such as a 27 G, 29 G, 30 G, or 31 G. The needle may be inserted into the tissue with consideration to the angle and orientation of the bevel, the depth of injection, and the quantity of material to be administered. The treatment may be injected into the tissue as a bolus, with for example a volume of about 10 μl to about 100 μl, about 10 μl to about 50 or about 20 μl to about 30 μl of product implanted at each injection site. After completion of each injection, the needle may be slowly withdrawn. When all implants have been completed the treated site may be gently massaged if required to enable the implant material to conform to the contour of the surrounding tissues. The number of treatments, the period between treatments and the amount of tropoelastin delivered at each treatment site will be adjusted based on the tissue area to be treated and the level of elasticity to be restored.

In some embodiments, methods of the disclosure increase glycosaminoglycan deposition in the soft tissue of a subject in need thereof. In some embodiments, increased glycosaminoglycan deposition comprises increased deposition of hyaluronic acid.

In some embodiments, increased glycosaminoglycan deposition in skin of a subject comprises endogenous glycosaminoglycan, i.e., glycosaminoglycan that is produced by cells at or proximal to the site of administration and not introduced by the composition itself In certain embodiments, the methods increase endogenous glycosaminoglycan deposition, such as increased endogenous hyaluronic acid deposition in the soft tissue. In further embodiments, glycosaminoglycan deposition is increased on the surface of cells of a papillary or upper reticular dermis in the skin.

In embodiments, glycosaminoglycan deposition is increased by about 2-fold, about 3-fold, about 4-fold, about 5-fold, about 6-fold, about 7-fold, about 8-fold, about 9-fold, or about 10-fold.

Methods of detecting glycosaminoglycan deposition are known in the art. For example, in embodiments, glycosaminoglycan deposition is detected by histologic evaluation of tissue biopsy.

In some embodiments, the disclosure provides methods of increasing hyaluronan synthase (e.g., hyaluronan synthase 1 (HAS1), hyaluronan synthase 2 (HAS2), and/or hyaluronan synthase 3 (HAS3)) expression in the skin of a subject of a subject in need thereof, the method comprising administering a composition comprising tropoelastin crosslinked to hyaluronic acid to the skin, wherein a ratio of tropoelastin:hyaluronic acid in the composition is about 2:1 or greater, and wherein hyaluronan synthase expression is increased as compared to a soft tissue administered an otherwise identical composition with a ratio of tropoelastin:hyaluronic acid less than 2:1. In some embodiments, the ratio of tropoelastin:hyaluronic acid in the composition is about 6:1 or greater, and hyaluronan synthase expression is increased as compared to a soft tissue administered an otherwise identical composition with a ratio of tropoelastin:hyaluronic acid less than 6:1. In some embodiments, hyaluronan synthase expression is increased by about 2-fold, about 3-fold, about 4-fold, about 5-fold, about 6-fold, about 7-fold, about 8-fold, about 9-fold, or about 10-fold relative to hyaluronan synthase expression in untreated skin.

Methods of evaluating hyaluronan synthase (e.g., hyaluronan synthase 1 (HAS1), hyaluronan synthase 2 (HAS2), and/or hyaluronan synthase 3 (HAS3)) expression in a soft tissue such as skin are well known in the art. In embodiments, hyaluronan synthase expression is measured at the level of hyaluronan synthase gene expression, such as by evaluating an mRNA level. In other embodiments, hyaluronan synthase expression is measured at the level of protein production.

### EXAMPLES

### Example 1

Tropoelastin increases moisture content, elastin content and glycosaminoglycan deposition in human skin explants and rat biopsy specimens.

### Tropoelastin and Hyaluronic Acid: Preparation and Formulations.

Recombinant human (rhTE) was produced in E. coli and purified as 60-kDa monomers as previously described (Martin S L, Vrhovski B, Weiss A S. Total synthesis and expression in Escherichia coli of a gene encoding human tropoelastin. Gene. 1995;154(2):159-166.) rhTE corresponded to amino acid residues 27-724 of GenBank entry AAC98394 isoform SHELdelta26A (Vrhovski B, Jensen S, Weiss A S. Coacervation characteristics of recombinant human tropoelastin. Eur. J. Biochem. 1997;250(1):92-98.). rhTE is produced according to current Good Manufacturing Practices (cGMP) code (Elastagen, Sydney, Australia). Hyaluronic acid was manufactured according to cGMP and sourced from HTL SAS (Javené, France).

The nine formulations prepared for the studies described herein are shown in Table 1. For the formulations of rhTE crosslinked with dHA, the dHA were produced as described in U.S. Pat. No. 9,611,312.

TABLE 1
Tropoelastin Formulations

| Formulation | Components |
|---|---|
| F1 | 10 mg/mL rhTE in monomer form in PBS |
| F2 | 10 mg/mL rhTE in monomer form mixed with 0.8% |
| | noncrosslinked HA in PBS |
| F3 | 2 mg/mL rhTE in monomer form mixed with 0.8% |
| | noncrosslinked HA in PBS |
| F4 | 4 mg/mL rhTE crosslinked with 0.8% dHA in PBS |
| F5 | 0.8% noncrosslinked HA |
| F6 | 30 mg/mL rhTE monomers mixed with HYC-24L+ |
| | (Juvéderm Ultra Plus XC; Allergan plc, Dublin, Ireland), |
| | final concentration of 12 mg/mL HA |
| F7 | 10 mg/mL rhTE monomers mixed with HYC-24L+ (final |
| | concentration of 12 mg/mL HA) |
| F8 | 30 mg/mL rhTE crosslinked with 0.5% dHA in PBS |
| F9 | 10 mg/mL rhTE crosslinked with 0.5% dHA in PBS |

### Ex Vivo Human Skin Explant Preparation.

Skin explants from a 63-year-old Caucasian female undergoing an abdominoplasty were cut into 15x20 mm rectangles and maintained in Explants Medium (BIO-EC; Longjumeau, France) at 37° C. and 5% CO2. On day zero (0), explants were injected either once with 50 μL of a test formulation (n=3 per group per time point for glycosaminoglycans [GAGs] analysis) or in a pattern of three injections of 50 μL of the test formulation separated by a distance of 5 mm (n=3 for corneometry analysis). Test formulations were: untreated control (NT), 12 mg/mL HA only, and formulations F6, F7, F8, and F9. On days 5 and 7, explants were collected for histology.

### Histological Evaluation of Glycosaminoglycans (GAGs) in Human Explants.

Explants were fixed in formol solution for 24 hours, dehydrated, embedded in paraffin, and cut into 5-μm sections. To detect acidic GAGs, slides were stained with Alcian Blue and Periodic Acid-Schiff (Mowry Method). Images were obtained using an Olympus (Shinjuku, Tokyo, Japan) BX43 microscope with a DP72 camera and the percent area of positive staining analyzed using Cell\hat{ } AD software.

### Evaluation of Skin Moisture Content.

To determine skin moisture content, a CM825 Corneometer® (Courage+Khazaka electronic; Köln, Germany) was applied to the explant surface at the center of the three injections. For each explant, ten measurements were taken on days 0, 1, 2, 5, and 7. Data are presented as the mean (SD) of the ten measurements from three explants per condition.

### Rat Protocols.

All animal protocols were approved by the Allergan Animal Care and Use Committee. Six-week-old male CD Hairless rats were obtained from Charles River Labs (Wilmington, Mass., USA) and housed for five days before starting experiments. Prior to injections, rats were anesthetized with 4% isoflurane and both flanks shaved. A 27 G ½-inch needle was used to inject 20 μL of a test formulation into the dermis. Test formulations were saline (control) and formulations F6, F7, F8, and F9 (n=8 per group per time point).

### Histologic Evaluation of Rat Skin Biopsies.

At 2 and 8 weeks, punch biopsies (8 mm) were collected, fixed in 10% neutral buffered formalin, embedded in paraffin, and sectioned. Immunohistochemical staining was performed using primary antibodies specific for rat elastin (Ab23748; Abcam, Cambridge, UK) and human elastin (MAB2503; Millipore, Burlington, Mass., USA) and anti-mouse (760-4814) and anti-rabbit (760-4815) secondary antibodies (Ventana Medical Systems, Tucson, Ariz., USA). Immunohistochemical staining was performed using the Ventana Discovery Benchmark Ultra autostainer system (Ventana Medical Systems) and imaged using a NanoZoomer (Hamamatsu Photonics, Hamamatsu City, Japan). Sections were also stained using hematoxylin and eosin (H&E) and images were graded from 0 to 5 based on severity for material spread, inflammation, and injection site fibrosis by a licensed veterinary pathologist who was blinded to treatment.

### Results

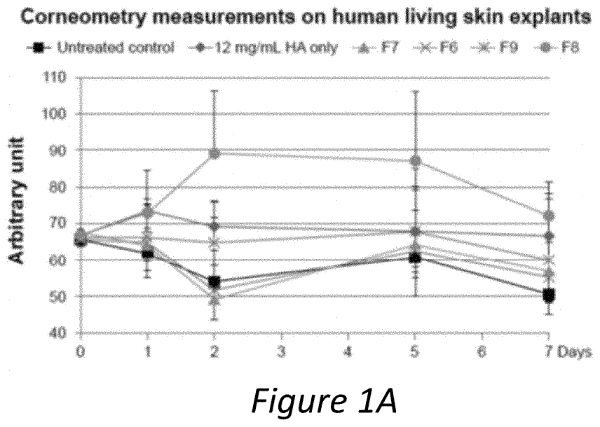

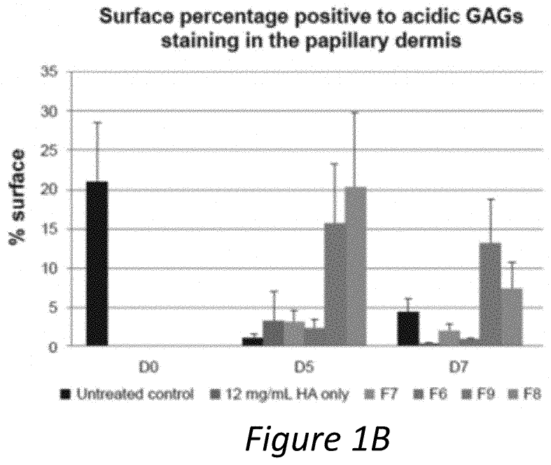

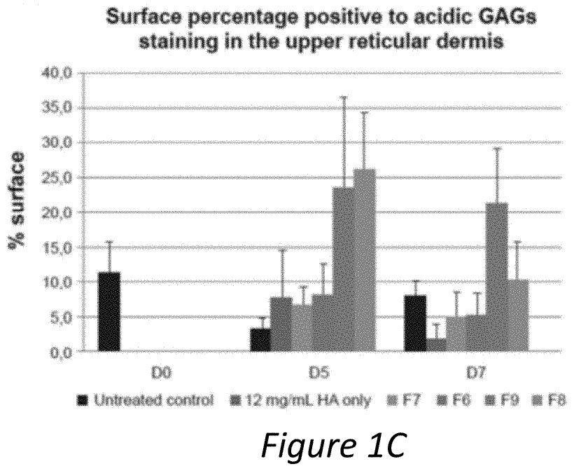

Compared with untreated controls, injection of F8 (30 mg rhTE crosslinked with 0.5% dHA, 6:1 ratio of tropoelastin to hyaluronic acid) significantly increased skin moisture content by 30.3% (P<0.01) at seven days after injection (FIG. 1A). This formulation tended to increase moisture content at days 2 and 5. This increase in moisture content was greater than that achieved with the 2:1 ratio of tropoelastin to hyaluronic acid, and formulations containing uncrosslinked rhTE monomer with HA (F6 and F7) had no effect on skin moisture content (FIG. 1A).

Immunohistochemical analysis of acidic GAGs revealed that intradermal injection of formulations produced from rhTE crosslinked with dHA (F8 and F9), but not the formulations of rhTE mixed with HYC-24L+ (F6 and F7), led to increased GAG staining in the papillary and upper reticular dermis at days 5 and 7 (FIGS. 1B and 1C).

Two weeks after intradermal injection, both the uncrosslinked (F6 and F7) and crosslinked (F8 and F9) formulations were detectable throughout the dermal layer of skin biopsies (Table 2). All test formulations resulted in minimal injection tract fibrosis and inflammation at 2 weeks; by 8 weeks, there was no observed inflammation and little evidence of injection tract fibrosis in all groups except for formulation F6 (30 mg/mL rhTE mixed with HYC-24L+; Table 2).

TABLE 2
Histologic Findings in Ex Vivo Human Skin Explant Preparation

| | | | | | | |
|---|---|---|---|---|---|---|
| | | | | | 12 | |
| | | | | | mg/mL | |
| Mean Severity Scores | F6 | F7 | F8 | F9 | HA | Saline |

Scores at Two Weeks

| | | | | | | |
|---|---|---|---|---|---|---|
| Injection tract fibrosis | 0.8 | 0.8 | 0.5 | 0.4 | 0.4 | 0.1 |
| Granulomatous inflammation | 0 | 0.3 | 0.6 | 0.8 | 0.1 | 0 |
| Material spread dermis | 3.0 | 3.3 | 2.1 | 2.5 | 2.1 | 0 |

Scores at Eight Weeks

| | | | | | | |
|---|---|---|---|---|---|---|
| Injection tract fibrosis | 1.4 | 0.5 | 0.4 | 0 | 0 | 0.1 |
| Granulomatous inflammation | 0 | 0 | 0 | 0 | 0 | 0 |
| Material spread dermis | 2.8 | 2.8 | 1.0 | 0.9 | 2.6 | 0 |

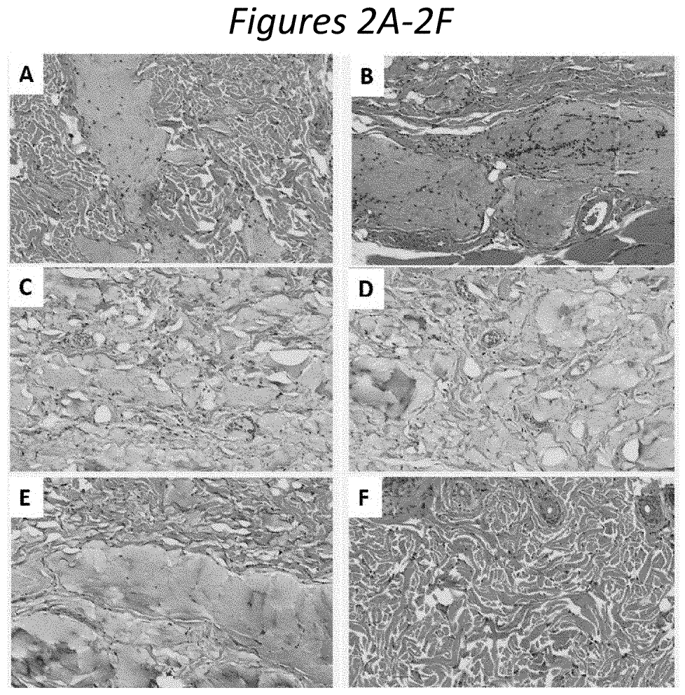

Histologic analysis of rat skin biopsies at two weeks revealed that the crosslinked formulations (F8 and F9) caused substantial cellular infiltration and material spread (FIGS. 2A and 2B). However, by eight weeks, less hydrogel was visible for crosslinked formulations compared with uncrosslinked formulations (Table 2). In contrast, injection of the uncrosslinked formulations (F6 and F7) resulted in noticeable material spread at both two and eight weeks (Table 2), with minimal cellular infiltration (FIG. 2C and 2D). Injection with 12 mg/mL HYC-24L+ (HA) alone (FIG. 2E) or saline (FIG. 2F) are shown for comparison.

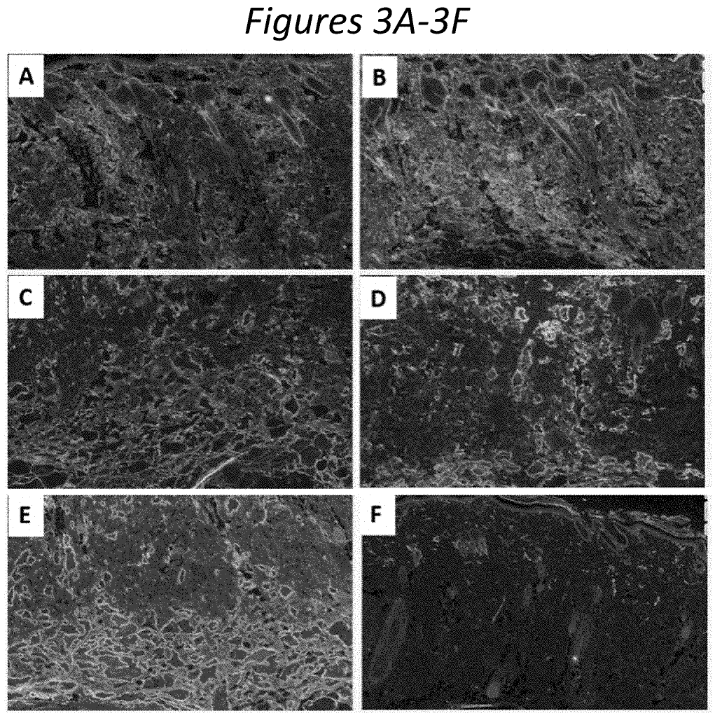

Further analysis of elastin composition revealed that injection of the rhTE+HA crosslinked formulations led to a stronger human elastin-positive signal than did the mixed material as well as colocalization of rat and human elastin-positive signals (FIGS. 3A and 3B), suggesting integration of added and new elastin. In contrast, injection of the uncrosslinked formulations displayed strong specific rat elastin staining surrounding the hydrogel and no colocalization of rat and human elastin staining within the tissue, similar to injections of HA alone (FIGS. 3C, 3D, and 3E). Injection with saline is shown in FIG. 3F.

### Example 2

### Tropoelastin Supports Organized Elastin and Collagen Production by Fibroblasts.

### Cell Culture and Microscopy.

Human dermal fibroblasts (C0045C; Thermo Fisher Scientific, Waltham, Mass., USA) were seeded on glass coverslips and grown in Dulbecco's Modified Eagle Medium (Life Technologies, Carlsbad, Calif., USA) supplemented with 10% fetal bovine serum (Life Technologies) and 1% penicillin-streptomycin (Sigma-Aldrich, St. Louis, Mo., USA). Cells were cultured for 24 days at 37° C. and 5% CO_{2,} and the media were changed every two to three days. To assess and facilitate collagen production, fibroblasts were incubated in the presence or absence of 50 μM L-ascorbic acid 2-phosphate sesquimagnesium salt hydrate (APM; Sigma-Aldrich). Cultures were treated with either 1 dose of filter-sterilized rhTE (formulation F 1; final media concentration, 0.25 mg/mL) on day 10, or one dose each on days 10 and 17. Elastin and collagen fibers were stained with primary rabbit polyclonal elastin antibody (α-hTE-17-F; gift from Dr. Dieter Reinhart, McGill University) and primary mouse monoclonal collagen antibody (COL-I; Sigma-Aldrich). Secondary antibodies (Alexa Fluor 568 goat anti-rabbit IgG and Alexa Fluor 488 goat anti-mouse IgG; Life Technologies) were used for visualization. Coverslips were mounted with Prolong Glass antifade mountant containing Nuc Blue stain to visualize nuclei (Thermo Fisher Scientific). Samples were visualized with a Nikon C2 confocal microscope (Tokyo, Japan) and images were produced using ImageJ software where Z-stacks were converted to maximum projection images of four equal sequential layers of the cell matrix.

### Results

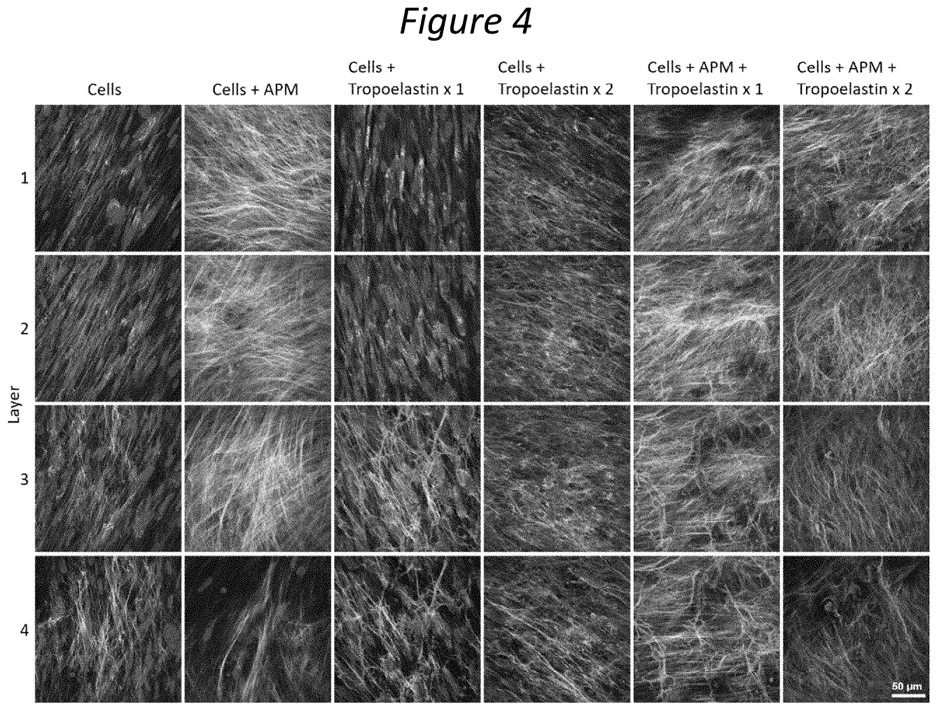

FIG. 4 shows four equal sequential layers through the cell-matrix culture, from the uppermost to lowest layer. Without rhTE and APM, fibroblasts displayed low levels of collagen and elastin synthesis. In the presence of APM, cells made ample collagen, which was predominantly confined to the top 75% of layers. When incubated with one dose of rhTE in the absence of APM, extensive networks of elastin fibers were found in the lower layers; two doses of rhTE extended the branched elastin network throughout the culture. In the presence of APM and rhTE, both fibrous collagen and branched elastin fibers were present; again, an elastin network formed at the base of the culture after one dose of rhTE or throughout the culture following two time-separated doses of rhTE (FIG. 4).

### Example 3

### Tropoelastin Stimulates Expression of Hyaluronan Synthase 1.

### 3D In Vitro Skin-Patch Model.

The Phenion® Full Thickness Skin Model (Henkel, Dusseldorf, Germany) was used according to the manufacturer's instructions. Skin patches (n=3 per test formulation) were cultured in Air-Liquid Interface (ALI) culture media in a 37° C. incubator supplemented with 5% CO_{2.} Skin patches were injected with a total volume of 50 μL of a test formulation at four points across the skin-patch surface. Test items were: control (positive: saline or 1% Triton X-100; negative: non-treated), and formulations F1, F2, F3, F4, and F5.

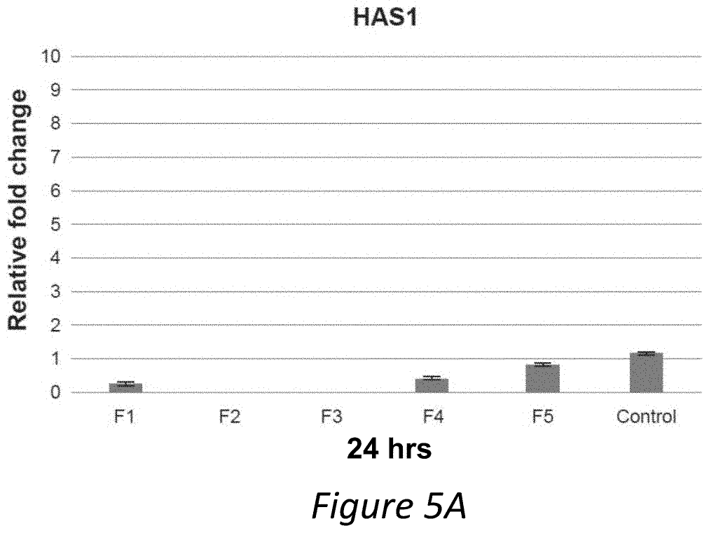

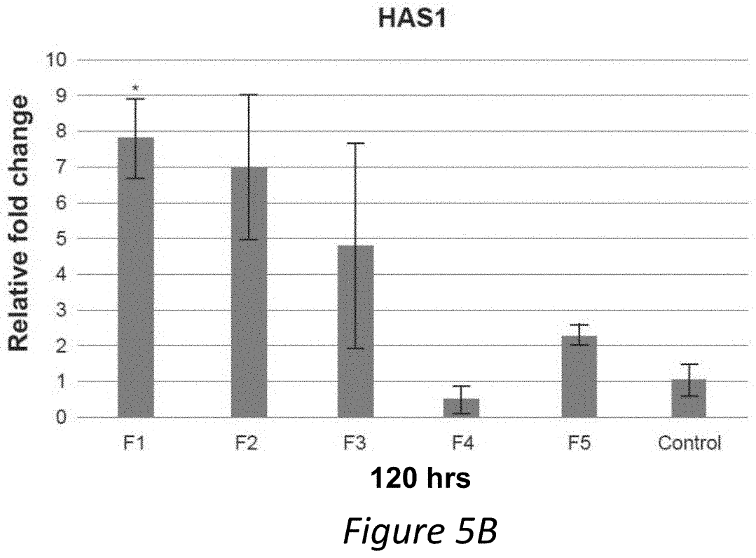

Intradermal injection of both free rhTE and rhTE+HA increased HAS1 mRNA at 120 hours (FIG. 5B). The effect of rhTE injection on HAS1 expression is delayed: levels of HAS1 mRNA are nearly undetectable at 24 hours but increased to a detectable level after 120 hours. In addition, the increase in HAS1 mRNA expression was proportional to the concentration of available rhTE implanted, with formulation F1 (containing the highest concentration of rhTE) having the greatest effect, followed by F2 and F3; a minimal effect was seen for F4 (the rhTE was crosslinked with dHA in a relatively high 2:1 dHA to rhTE ratio) and F5 (HA alone).

### Example 4

### Tropoelastin Stimulates GAG Deposition by Dermal Fibroblasts

### Materials and Methods.

Human dermal fibroblasts were sourced from a neonatal male (Thermo Fisher, C0045C, foreskin) and a 51-year-old male (Sigma, 142BR Lot 05/H/014). Dermal fibroblasts (1×10⁴ cells) were seeded directly in the wells of 12 well tissue culture plates in 4 ml fresh media (DMEM (Life Tech) containing 10% (v/v) fetal bovine serum (FBS; Life Tech) and 1% (v/v) Pen/Strep (Sigma)). Cells were cultured at 37° C., 5% CO₂ with media 4 changes every 2-3 days. On day 10 of culture, rhTE (15 mg/ml in phosphate buffered saline (PBS); filter-sterilized; 1.5 mg/well), HA (0.5% w/v in PBS; filter-sterilized; 50 μl/well), or both, was added to the wells and the cells were cultured for a further 7 days.

GAG deposition by dermal fibroblasts following 17 days of culture and 7 days after the addition of HA and/or rhTE was assessed using Alcian blue staining (Lopez-Gonzalez, et al. (2017) J. Dent. Res. 96, 832.). Cultured cells and deposited ECM were fixed with 4% paraformaldehyde in PBS for 1 hour at room temperature, rinsed three times in PBS then stained overnight at room temperature with Alcian blue solution (1% in 3% acetic acid, pH 2.5; Sigma B8438). Samples were then rinsed twice with 3% acetic acid, twice with H₂O and the stained cultures were photographed. The Alcian blue stain was extracted from the cell/ECM matrix in 6 M guanidine hydrochloride (Sigma G4505) for 6 hours and the absorbance of the resulting solution was measured at 630 nm in a Tecan plate reader.

### Results.

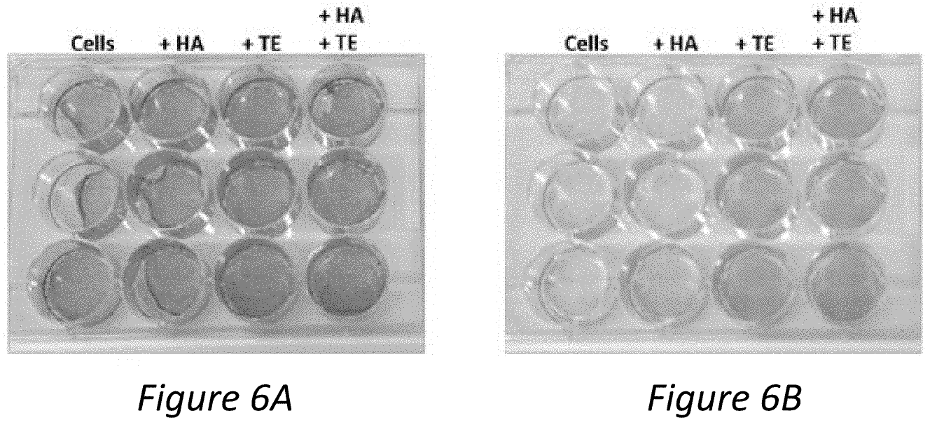

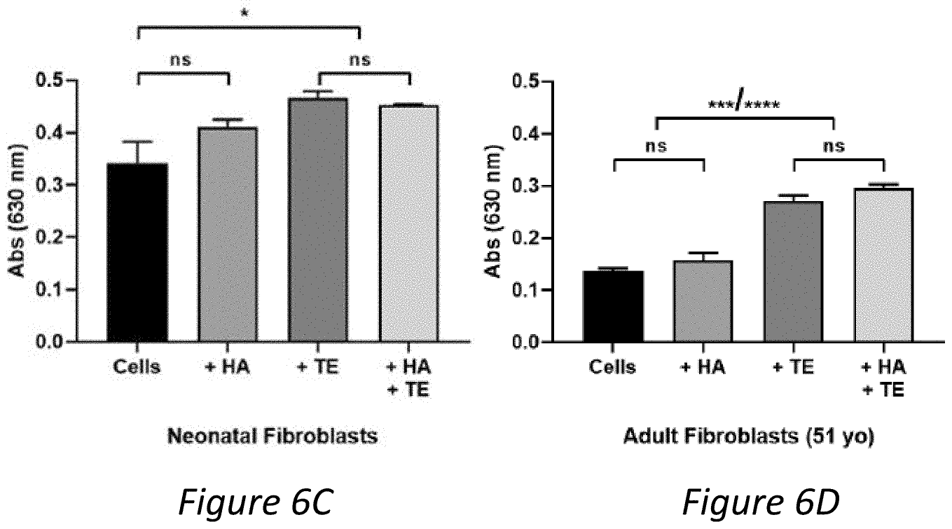

Tropoelastin supplementation of dermal fibroblast cultures resulted in a significant increase in ECM accumulation of GAGs by neonatal fibroblasts compared to un-supplemented neonatal fibroblasts (FIGS. 6A and 6C) and by adult fibroblasts compared to both un-supplemented and HA-supplemented adult fibroblasts (FIGS. 6B and 6D). Addition of tropoelastin restored GAG accumulation in adult fibroblast cultures to a level similar to that seen in neonatal fibroblast cultures.

### Illustration of Subject Technology as Clauses

Various examples of aspects of the disclosure are described as numbered clauses (1, 2, 3, etc.) for convenience. These are provided as examples, and do not limit the subject technology.

Clause 1. A method of treating a soft tissue condition in a subject in need thereof, the method comprising: administering a composition comprising tropoelastin and optionally hyaluronic acid to a soft tissue of the subject, wherein the composition increases a moisture content in the soft tissue of the subject.

Clause 2. A method of treating a soft tissue condition in a subject in need thereof, the method comprising: administering a composition comprising tropoelastin crosslinked to hyaluronic acid to a soft tissue of the subject, wherein the composition increases a moisture content in the soft tissue of the subject as compared to an otherwise identical composition where the tropoelastin is not crosslinked to the hyaluronic acid.

Clause 3. The method of Clause 2, wherein the tropoelastin is crosslinked to the hyaluronic acid without a crosslinker.

Clause 4. The method of Clause 2, wherein the tropoelastin is crosslinked to the hyaluronic acid via at least one intermolecular cross-linkage comprising an amide bond between an amine of the tropoelastin and a carboxyl group of the hyaluronic acid.

Clause 5. The method of Clause 1 or 2, wherein the tropoelastin comprises human tropoelastin.

Clause 6. The method of Clause 1 or 2, wherein the tropoelastin comprises recombinant tropoelastin.

Clause 7. The method of Clause 1 or 2, wherein the tropoelastin comprises recombinant human tropoelastin.

Clause 8. The method of Clause 1 or 2, wherein the tropoelastin comprises tropoelastin monomers.

Clause 9. The method of any one of the preceding Clauses, wherein the hyaluronic acid is derivatized hyaluronic acid.

Clause 10. The method of any one of the preceding Clauses, wherein the tropoelastin is present in an amount of about 1 mg/ml to about 100 mg/mL.

Clause 11. The method of any one of the preceding Clauses, wherein the tropoelastin is present in an amount of about 10 to about 50 mg/mL.

Clause 12. The method of Clause 11, wherein the tropoelastin is present in an amount of about 30 mg/mL.

Clause 13. The method of any one of the preceding Clauses, wherein the tropoelastin is crosslinked with about 0.1% to about 5% hyaluronic acid.

Clause 14. The method of Clause 13, wherein the tropoelastin is crosslinked with about 0.5% hyaluronic acid.

Clause 15. The method of any one of the preceding Clauses, wherein hyaluronic acid is present in an amount of about 1 mg/mL to about 15 mg/mL.

Clause 16. The method of Clause 2, wherein a ratio of tropoelastin:hyaluronic acid is about 2:1, about 3:1, about 4:1, about 5:1, about 6:1, about 7:1, about 8:1, about 9:1, or about 10:1.

Clause 17. The method of Clause 2, wherein a ratio of tropoelastin:hyaluronic acid is about 2:1 or greater, and wherein composition increases a moisture content in the soft tissue of the subject as compared to a soft tissue administered an otherwise identical composition with a ratio of tropoelastin:hyaluronic acid less than the ratio of tropoelastin:hyaluronic acid in the composition.

Clause 18. The method of Clause 17, wherein the amount of hyaluronic acid is less than or equal to about 5 mg/mL, and wherein the composition increases a moisture content in the soft tissue of the subject as compared to an otherwise identical composition with greater than about 5 mg/mL hyaluronic acid.

Clause 19. The method of Clause 2, wherein a ratio of tropoelastin:hyaluronic acid is about 6:1 or greater, and wherein the composition increases a moisture content in the soft tissue of the subject as compared to an otherwise identical composition with a ratio of tropoelastin:hyaluronic acid less than 6:1.

Clause 20. The method of Clause 19, wherein the amount of hyaluronic acid is less than or equal to about 5 mg/mL, and wherein the composition increases a moisture content in the soft tissue of the subject as compared to an otherwise identical composition with greater than about 5 mg/mL hyaluronic acid.

Clause 21. The method of any one of the preceding Clauses, wherein the soft tissue is skin.

Clause 22. The method of Clause 21, wherein the step of administering the composition comprises an injection of the composition into the skin.

Clause 23. The method of Clause 21, wherein the step of administering the composition comprises an intradermal injection of the composition into a dermis.

Clause 24. The method of Clause 21, wherein the step of administering the composition comprises an injection of the composition into a hypodermis.

Clause 25. The method of Clause 21, wherein the step of administering the composition comprises an injection of the composition into a sub-dermis.

Clause 26. The method of any one of the preceding Clauses, wherein the composition is an injectable composition.

Clause 27. The method of any one of the preceding Clauses, wherein the step of administering the composition increases glycosaminoglycan deposition in the tissue.

Clause 28. The method of Clause 27, wherein the step of administering the composition increases endogenous glycosaminoglycan deposition in the tissue.

Clause 29. The method of Clause 27, wherein the step of administering the composition increases hyaluronic acid deposition in the tissue.

Clause 30. The method of Clause 29, wherein the hyaluronic acid is endogenous.

Clause 31. The method of Clause 27, wherein glycosaminoglycan deposition is increased on the surface of cells of a papillary or upper reticular dermis in the skin.

Clause 32. The method of Clause 27, wherein the glycosaminoglycan deposition is increased between about 1-fold and about 15-fold.

Clause 33. The method of Clause 27, wherein the glycosaminoglycan deposition is increased between about 2-fold and about 10-fold.

Clause 34. The method of Clause 27, wherein the glycosaminoglycan deposition is increased by about 2-fold, about 3-fold, about 4-fold, about 5-fold, about 6-fold, about 7-fold, about 8-fold, about 9-fold, or about 10-fold.

Clause 35. The method of any one of the preceding Clauses, wherein the moisture content of the soft tissue is increased between about 10% to about 80%.

Clause 36. The method of any one of the preceding Clauses, wherein the moisture content of the soft tissue is increased between about 20% to about 50%.

Clause 37. The method of any one of the preceding Clauses, wherein the moisture content of the soft tissue is increased by about 20%, about 25%, about 30%, about 35%, about 40%, about 45%, or about 50%.

Clause 38. The method of any one of the preceding Clauses, wherein the step of administering the composition stimulates collagen synthesis in the soft tissue.

Clause 39. The method of any one of the preceding Clauses, wherein the step of administering the composition stimulates elastin synthesis in the soft tissue.

Clause 40. The method of any one of the preceding Clauses, wherein the step of administering the composition stimulates collagen and elastin synthesis in the soft tissue.

Clause 41. The method of any one of the preceding Clauses, wherein the step of administering the composition leads to cellular infiltration at the site of administration.

Clause 42. The method of any one of the preceding Clauses, wherein the step of administering the composition increases elasticity of the tissue.

Clause 43. The method of any one of the preceding Clauses, wherein the soft tissue is skin.

Clause 44. The method of clause 43, wherein the skin is very dry skin, dry skin, or hydrated skin before administration of the composition.

Clause 45. The method of clause 44, wherein the skin has a capacitance of less than about 90 a.u., less than about 85 a.u., less than about 80 a.u., less than about 75 a.u., less than about 70 a.u., less than about 65 a.u., less than about 60 a.u., less than about 55 a.u., less than about 50 a.u., less than about 45 a.u., less than about 40 a.u., less than about 35 a.u., less than about 30 a.u., less than about 25 a.u., less than about 20 a.u., less than about 15 a.u., less than about 10 a.u., or less than about 5 a.u. before administration of the composition.

Clause 46. A method of increasing a moisture content in a soft tissue of a subject in need thereof, the method comprising: providing a subject having a soft tissue with a first moisture content; and administering a composition comprising tropoelastin crosslinked to hyaluronic acid to the soft tissue in an amount effective to produce a second moisture content in the soft tissue, wherein a ratio of tropoelastin:hyaluronic acid in the composition is about 2:1 or greater, and wherein the second moisture content is increased as compared to a soft tissue administered an otherwise identical composition with a ratio of tropoelastin:hyaluronic acid less than the ratio of tropoelastin:hyaluronic acid in the composition.

Clause 47. The method of Clause 46, wherein the ratio of tropoelastin:hyaluronic acid in the composition is about 6:1, and wherein the second moisture content is increased as compared to a soft tissue administered an otherwise identical composition with a ratio of tropoelastin:hyaluronic acid less than about 6:1.

Clause 48. The method of any one of Clauses 46 to 47, wherein the second moisture content is between about 10% to about 80% higher than the first moisture content.

Clause 49. The method of any one of Clauses 46 to 48, wherein the second moisture content is between about 20% to about 50% higher than the first moisture content.

Clause 50. The method of any one of Clauses 46 to 49, wherein second moisture content is about 20%, about 25%, about 30%, about 35%, about 40%, about 45%, or about 50% higher than the first moisture content.

Clause 51. The method of any one of Clauses 46 to 50, wherein the second moisture content remains higher than the first moisture content for at least 4 weeks, at least 8 weeks, at least 12 weeks, at least 24 weeks, or at least 48 weeks.

Clause 52. The method of any one of Clauses 46 to 51, wherein the tropoelastin is crosslinked to the hyaluronic acid without a crosslinker.

Clause 53. The method of Clause 52, wherein the tropoelastin is crosslinked to the hyaluronic acid via at least one intermolecular cross-linkage comprising an amide bond between an amine of the tropoelastin and a carboxyl group of the hyaluronic acid.

Clause 54. The method of any one of Clauses 46 to 53, wherein the tropoelastin is human tropoelastin.

Clause 55. The method of any one of Clauses 46 to 53, wherein the tropoelastin is recombinant tropoelastin.

Clause 56. The method of any one of Clauses 46 to 53, wherein the tropoelastin is recombinant human tropoelastin.

Clause 57. The method of any one of Clauses 46 to 56, wherein the tropoelastin comprises tropoelastin monomers.

Clause 58. The method of any one of Clauses 46 to 57, wherein the hyaluronic acid is derivatized hyaluronic acid.

Clause 59. The method of any one of Clauses 46 to 58, wherein the tropoelastin is present in an amount of about 1 mg/ml to about 100 mg/mL.

Clause 60. The method of any one of Clauses 46 to 59, wherein the tropoelastin is crosslinked with about 0.1% to about 5% hyaluronic acid.

Clause 61. The method of any one of Clauses 46 to 60, wherein hyaluronic acid is present in an amount of about 1 mg/mL to about 15 mg/mL.

Clause 62. The method of Clause 61, wherein the hyaluronic acid is present in an amount of about 10 mg/mL or less, about 5 mg/mL or less, about 4 mg/mL or less, about 3 mg/mL or less, about 2 mg/mL or less, or about 1 mg/mL or less.

Clause 63. The method of any one of Clauses 46 to 62, wherein the ratio of tropoelastin:hyaluronic acid is about 2:1, about 3:1, about 4:1, about 5:1, about 6:1, about 7:1, about 8:1, about 9:1, or about 10:1.

Clause 64. The method of Clause 63, wherein the ratio of tropoelastin:hyaluronic acid is about 2:1 or greater.

Clause 65. The method of any one of Clauses 46 to 64, wherein the soft tissue is skin.

Clause 66. The method of Clause 65, wherein the step of administering the composition comprises an injection of the composition into the skin.

Clause 67. The method of Clause 65, wherein the step of administering the composition comprises an intradermal injection into a dermis.

Clause 68. The method of Clause 65, wherein the step of administering the composition comprises an injection into a hypodermis.

Clause 69. The method of Clause 65, wherein the step of administering the composition comprises an injection into a sub-dermis.

Clause 70. The method of Clause 46, wherein the subject has a soft tissue condition selected from the group consisting of: a facial wrinkle, a fine line, thinning skin, aging skin, scar tissue, and a skin depression.

Clause 71. The method of Clause 46, wherein the composition is an injectable composition.

Clause 72. A method of repairing a soft tissue, the method comprising: providing a soft tissue in need of repair; and contacting the soft tissue with a composition comprising tropoelastin crosslinked to hyaluronic acid, wherein the method increases a moisture content in the tissue.

Clause 73. The method of Clause 72, wherein the soft tissue comprises fibroblast cells.

Clause 74. The method of any one of Clauses 72 to 73, wherein the tropoelastin is crosslinked to hyaluronic acid without a crosslinker.

Clause 75. The method of Clause 74, wherein the tropoelastin is crosslinked to the hyaluronic acid via at least one intermolecular cross-linkage comprising an amide bond between an amine of the tropoelastin and a carboxyl group of the hyaluronic acid.

Clause 76. The method of any one of Clauses 72 to 75, wherein the tropoelastin is recombinant tropoelastin.

Clause 77. The method of any one of Clauses 72 to 75, wherein the tropoelastin is recombinant human tropoelastin.

Clause 78. The method of any one of Clauses 72 to 77, wherein the tropoelastin comprises tropoelastin monomers.

Clause 79. The method of any one of Clauses 72 to 78, wherein the hyaluronic acid is derivatized hyaluronic acid.

Clause 80. The method of any one of Clauses 72 to 79, wherein the tropoelastin is present in an amount of about 1 mg/ml to about 100 mg/mL.

Clause 81. The method of any one of Clauses 72 to 81, wherein the tropoelastin is present in an amount of about 10 to about 50 mg/mL.

Clause 82. The method of Clause 81, wherein the tropoelastin is present in an amount of about 30 mg/mL.

Clause 83. The method of any one of Clauses 72 to 82, wherein the tropoelastin is crosslinked with about 0.1% to about 5% hyaluronic acid.

Clause 84. The method of any one of Clauses 72 to 83, wherein the tropoelastin is crosslinked with about 0.5% hyaluronic acid.

Clause 85. The method of any one of Clauses 72 to 84, wherein hyaluronic acid is present in an amount of about 1 mg/mL to about 15 mg/mL.

Clause 86. The method of any one of Clauses 72 to 85, wherein the hyaluronic acid is present in an amount of about 10 mg/mL or less, about 5 mg/mL or less, about 4 mg/mL or less, about 3 mg/mL or less, about 2 mg/mL or less, or about 1 mg/mL or less.

Clause 87. The method of any one of Clauses 72 to 86, wherein the ratio of tropoelastin:hyaluronic acid is about 2:1 or greater.

Clause 88. The method of any one of Clauses 72 to 87, wherein the moisture content is increased between about 10% to about 80%.

Clause 89. The method of any one of Clauses 72 to 88, wherein the step of contacting the soft tissue with the composition increases deposition of glycosaminoglycan.

Clause 90. A method of increasing glycosaminoglycan deposition in skin of a subject in need thereof, the method comprising administering a composition comprising tropoelastin crosslinked to hyaluronic acid to the skin, wherein a ratio of tropoelastin:hyaluronic acid in the composition is about 2:1 or greater, and wherein glycosaminoglycan deposition is increased as compared to a soft tissue administered an otherwise identical composition with a ratio of tropoelastin:hyaluronic acid less than 2:1.

Clause 91. The method of Clause 90, wherein the step of administering the composition increases glycosaminoglycan deposition on the surface of cells of a papillary or upper reticular dermis in the skin.

Clause 92. The method of Clause 91, wherein the glycosaminoglycan deposition is increased between about 1-fold and about 15-fold.

Clause 93. The method of any one of Clauses 91 or 92, wherein the glycosaminoglycan deposition is increased between about 2-fold and about 10-fold.

Clause 94. The method of any one of Clauses 91 to 93, wherein the glycosaminoglycan deposition is increased by about 2-fold, about 3-fold, about 4-fold, about 5-fold, about 6-fold, about 7-fold, about 8-fold, about 9-fold, or about 10-fold.

Clause 95. The method of any one of Clauses 90 to 94, wherein the tropoelastin is crosslinked to hyaluronic acid without a crosslinker.

Clause 96. The method of Clause 95, wherein the tropoelastin is crosslinked to the hyaluronic acid via at least one intermolecular cross-linkage comprising an amide bond between an amine of the tropoelastin and a carboxyl group of the hyaluronic acid.

Clause 97. The method of any one of Clauses 90 to 96, wherein the tropoelastin is recombinant human tropoelastin.

Clause 98. The method of any one of Clauses 90 to 97, wherein the tropoelastin is present in an amount of about 1 mg/ml to about 100 mg/mL.

Clause 99. The method of any one of Clauses 90 to 98, wherein the tropoelastin is crosslinked with about 0.1% to about 5% hyaluronic acid.

Clause 100. The method of any one of Clauses 90 to 99, wherein hyaluronic acid is present in an amount of about 1 mg/mL to about 15 mg/mL.

Clause 101. A method of increasing hyaluronan synthase expression in the skin of a subject, the method comprising administering a composition comprising tropoelastin crosslinked to hyaluronic acid to the skin, wherein a ratio of tropoelastin:hyaluronic acid in the composition is about 2:1 or greater, and wherein hyaluronan synthase expression is increased as compared to a soft tissue administered an otherwise identical composition with a ratio of tropoelastin:hyaluronic acid less than 2:1.

Clause 102. The method of Clause 101, wherein the tropoelastin is crosslinked to hyaluronic acid without a crosslinker.

Clause 103. The method of any one of Clauses 101 or 102, wherein the tropoelastin is crosslinked to the hyaluronic acid via at least one intermolecular cross-linkage comprising an amide bond between an amine of the tropoelastin and a carboxyl group of the hyaluronic acid.

Clause 104. The method of any one of Clauses 101 to 103, wherein hyaluronan synthase expression is increased by about 2-fold, about 3-fold, about 4-fold, about 5-fold, about 6-fold, about 7-fold, about 8-fold, about 9-fold, or about 10-fold relative to hyaluronan synthase expression in untreated skin.

Clause 105. The method of any one of Clauses 101 to 104, wherein hyaluronan synthase mRNA expression is increased.

Clause 106. The method of any one of Clauses 101 to 104, wherein hyaluronan synthase protein expression is increased.

Clause 107. A method of treating a soft tissue condition in a subject in need thereof, the method comprising: administering a composition comprising tropoelastin crosslinked to hyaluronic acid to a soft tissue of the subject, wherein the composition increases a moisture content in the soft tissue of the subject as compared to an otherwise identical composition where the tropoelastin is not crosslinked to the hyaluronic acid, wherein a ratio of tropoelastin:hyaluronic acid in the composition is about 2:1 or greater, and wherein the increased moisture content is increased as compared to a soft tissue administered an otherwise identical composition with a ratio of tropoelastin:hyaluronic acid less than the ratio of tropoelastin:hyaluronic acid in the composition.

Clause 108. A method of treating a soft tissue condition in a subject in need thereof, the method comprising: administering a composition comprising tropoelastin crosslinked to hyaluronic acid to a soft tissue of the subject, wherein a ratio of tropoelastin:hyaluronic acid in the composition is about 2:1 or greater, and wherein the composition increases a moisture content in the soft tissue of the subject as compared to a soft tissue administered an otherwise identical composition with a ratio of tropoelastin:hyaluronic acid less than the ratio of tropoelastin:hyaluronic acid in the composition.

Clause 109. A method of treating a soft tissue condition in a subject in need thereof, the method comprising: administering a composition comprising tropoelastin crosslinked to hyaluronic acid to a soft tissue of the subject, wherein a ratio of tropoelastin:hyaluronic acid in the composition is about 3:1 or greater, and wherein the composition increases a moisture content in the soft tissue of the subject as compared to a soft tissue administered an otherwise identical composition with a ratio of tropoelastin:hyaluronic acid less than the ratio of tropoelastin:hyaluronic acid in the composition. a soft tissue administered an otherwise identical composition with a ratio of tropoelastin:hyaluronic acid less than the ratio of tropoelastin:hyaluronic acid in the composition.

Clause 110. A method of treating a soft tissue condition in a subject in need thereof, the method comprising: administering a composition comprising tropoelastin crosslinked to hyaluronic acid to a soft tissue of the subject, wherein a ratio of tropoelastin:hyaluronic acid in the composition is about 4:1 or greater, and wherein the composition increases a moisture content in the soft tissue of the subject as compared to a soft tissue administered an otherwise identical composition with a ratio of tropoelastin:hyaluronic acid less than the ratio of tropoelastin:hyaluronic acid in the composition.

Clause 111. A method of treating a soft tissue condition in a subject in need thereof, the method comprising: administering a composition comprising tropoelastin crosslinked to hyaluronic acid to a soft tissue of the subject, wherein a ratio of tropoelastin:hyaluronic acid in the composition is about 5:1 or greater, and wherein the composition increases a moisture content in the soft tissue of the subject as compared to a soft tissue administered an otherwise identical composition with a ratio of tropoelastin:hyaluronic acid less than the ratio of tropoelastin:hyaluronic acid in the composition.

Clause 112. A method of treating a soft tissue condition in a subject in need thereof, the method comprising: administering a composition comprising tropoelastin crosslinked to hyaluronic acid to a soft tissue of the subject, wherein a ratio of tropoelastin:hyaluronic acid in the composition is about 6:1 or greater, and wherein the composition increases a moisture content in the soft tissue of the subject as compared to a soft tissue administered an otherwise identical composition with a ratio of tropoelastin:hyaluronic acid less than the ratio of tropoelastin:hyaluronic acid in the composition.

Clause 113. A method of treating a soft tissue condition in a subject in need thereof, the method comprising: administering a composition comprising tropoelastin crosslinked to hyaluronic acid to a soft tissue of the subject, wherein a ratio of tropoelastin:hyaluronic acid in the composition is about 7:1 or greater, and wherein the composition increases a moisture content in the soft tissue of the subject as compared to a soft tissue administered an otherwise identical composition with a ratio of tropoelastin:hyaluronic acid less than the ratio of tropoelastin:hyaluronic acid in the composition.

Clause 114. A method of treating a soft tissue condition in a subject in need thereof, the method comprising: administering a composition comprising tropoelastin crosslinked to hyaluronic acid to a soft tissue of the subject, wherein a ratio of tropoelastin:hyaluronic acid in the composition is about 8:1 or greater, and wherein the composition increases a moisture content in the soft tissue of the subject as compared to a soft tissue administered an otherwise identical composition with a ratio of tropoelastin:hyaluronic acid less than the ratio of tropoelastin:hyaluronic acid in the composition.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. In addition, it is not necessary for a method to address every problem that is solvable (or possess every advantage that is achievable) by different embodiments of the disclosure in order to be encompassed within the scope of the disclosure. The use herein of “can” and derivatives thereof shall be understood
in the sense of “possibly” or “optionally” as opposed to an affirmative capability.

1-51. (canceled)

52. A method of treating a soft tissue condition in a subject in need thereof, the method comprising: administering a composition comprising tropoelastin crosslinked to hyaluronic acid to a soft tissue of the subject, wherein the composition increases a moisture content in the soft tissue of the subject as compared to an otherwise identical composition where the tropoelastin is not crosslinked to the hyaluronic acid.

53. The method of claim 52, wherein the tropoelastin is crosslinked to the hyaluronic acid without a crosslinker.

54. The method of claim 53, wherein the tropoelastin is crosslinked to the hyaluronic acid via at least one intermolecular cross-linkage comprising an amide bond between an amine of the tropoelastin and a carboxyl group of the hyaluronic acid.

55. The method of claim 52, wherein the tropoelastin comprises human tropoelastin.

56. The method of claim 52, wherein the tropoelastin comprises recombinant tropoelastin.

57. The method of claim 52, wherein the tropoelastin comprises recombinant human tropoelastin.

58. The method of claim 52, wherein the tropoelastin is present in an amount of about 1 mg/ml to about 100 mg/mL.

59. The method of claim 52, wherein the tropoelastin is present in an amount of about 10 to about 50 mg/mL.

60. The method of claim 59, wherein the tropoelastin is present in an amount of about 30 mg/mL.

61. The method of claim 52, wherein the hyaluronic acid is derivatized hyaluronic acid.

62. The method of claim 52, wherein the tropoelastin is crosslinked with about 0.1% to about 5% hyaluronic acid.

63. The method of claim 62, wherein the tropoelastin is crosslinked with about 0.5% hyaluronic acid.

64. The method of claim 52, wherein the soft tissue is skin.

65. The method of claim 64, wherein the skin is very dry skin, dry skin, or hydrated skin before administration of the composition.

66. The method of claim 64, wherein the soft tissue condition is selected from the group consisting of: a facial wrinkle, a fine line, thinning skin, aging skin, scar tissue, and a skin depression.

67. The method of claim 64, wherein the step of administering the composition comprises an intradermal injection of the composition into a dermis.

68. The method of claim 52, wherein the step of administering the composition increases glycosaminoglycan deposition in the tissue.

69. The method of claim 52, wherein the moisture content of the soft tissue is increased between about 10% to about 80%.

70. The method of claim 52, wherein the moisture content of the soft tissue is increased between about 20% to about 50%.

71. The method of claim 52, wherein the moisture content of the soft tissue is increased by about 20%, about 25%, about 30%, about 35%, about 40%, about 45%, or about 50%.

72. A method of increasing infiltration of cells into a soft tissue in a subject in need thereof, the method comprising: administering a composition comprising tropoelastin crosslinked to hyaluronic acid to the soft tissue of the subject, wherein the composition results in an enhancement of one or more of the following as compared to an otherwise identical composition where the tropoelastin is not crosslinked to the hyaluronic acid:
i. increases infiltration of cells into the soft tissue of the subject;
ii. increases formation of elastin fibers;
iii. increases formation of new collagen fibers;
iv. formation of a new matrix at a site of administration;
v. increases elastin and collagen fiber formation;
vi. improving skin composition;
vii. improving skin brightness;
viii. improving skin elasticity; and
ix. increases extracellular matrix proteins at the site of administration.

73. A composition for intradermal injection comprising tropoelastin crosslinked to hyaluronic acid, wherein the tropoelastin is present in an amount of about 30 mg/mL and the tropoelastin is crosslinked with about 0.5% hyaluronic acid, wherein the tropoelastin is crosslinked to the hyaluronic acid via at least one intermolecular cross-linkage comprising an amide bond between an amine of the tropoelastin and a carboxyl group of the hyaluronic acid in the absence of a crosslinker.
